(19) United States
(12) Patent Application Publication
(10) Pub. No.: US 2026/0215920 A1
(43) Pub. Date: Jul. 30, 2026

(54) GUIDE FLEX SYSTEM FOR DELIVERY HANDLE FOR PROSTHETIC VALVE DELIVERY SYSTEMS

(71) Applicant: MiRus LLC, Marietta, GA (US)

(72) Inventors: Jay Yadav, Atlanta, GA (US); Ken Holloway, Marietta, GA (US); Miheer Joshi, Marietta, GA (US); Angela Rothfuss, Marietta, GA (US); Jorge Jimenez, Marietta, GA (US)

(21) Appl. No.: 19/574,544

(22) Filed: Mar. 23, 2026

Related U.S. Application Data
(63) Continuation-in-part of application No. 18/904,796, filed on Oct. 2, 2024.
Application No. 18/904,796 is a continuation-in-part of application No. 29/851,277, filed on Aug. 26, 2022, now Pat. No. D1087336.
(63) Continuation-in-part of application No. 30/021,457, filed on Sep. 4, 2025.
(63) Continuation-in-part of application No. 30/021,437, filed on Sep. 4, 2025.
(60) Provisional application No. 63/541,938, filed on Oct. 2, 2023.
(60) Provisional application No. 63/876,975, filed on Sep. 6, 2025.
(60) Provisional application No. 63/877,017, filed on Sep. 6, 2025.
(60) Provisional application No. 63/908,051, filed on Oct. 30, 2025.

(51) Int. Cl. A61F 2/24; A61M 25/00; A61M 25/01; A61M 25/06

(52) U.S. Cl. CPC A61F 2/2433; A61M 25/0045; A61M 25/0136; A61M 25/0144; A61M 25/0147; A61M 25/0662; A61M 2025/0004; A61M 2025/015; A61M 2205/0216; A61M 2205/583

(57) ABSTRACT

A delivery system that facilitates the delivery of an expandable medical device to a treatment site, and wherein the delivery system includes a delivery handle having housing that has an inner core that extends linearly between a proximal end and a distal end of the housing, and wherein the inner core receives a flexible catheter, and wherein the delivery handle includes a flex adjustment mechanism that is configured to controllably bend the distal region of the flexible catheter and/or balloon catheter; and wherein the flex adjustment mechanism includes a flex knob that is coupled to the housing, a flex insert and/or flex actuator that are movable by the flex knob, a wire member that is connected to the flex insert and/or flex actuator, and an optional flex indicator that provides information on the amount of bending of the flexible catheter and/or balloon catheter by the flex adjustment mechanism.

### REFERENCED APPLICATIONS

The present application is a continuation-in-part of U.S. patent application Ser. No. 18/904,796 filed Oct. 2, 2024, which in turn claims the benefit of U.S. Provisional Patent Application Ser. No. 63/541,938 filed on Oct. 2, 2023 and entitled, “Delivery Handle Arrangement for Prosthetic Valve Delivery Systems”, the disclosures of which are fully incorporated herein by reference.

The present application is a continuation-in-part of U.S. patent application Ser. No. 18/904,796 filed Oct. 2, 2024, which in turn is a continuation-in-part of U.S. application Ser. No. 29/851,277 filed Aug. 26, 2022 and entitled, “Delivery Handle Device”, the disclosures of which are fully incorporated herein by reference.

The present application is a continuation-in-part of United States Design application Ser. Nos. 30/021,457 filed Sep. 4, 2025 and Ser. No. 30/021,437 filed Sep. 4, 2025, both of which are fully incorporated herein by reference.

This application is claims priority to U.S. Provisional Application Ser. No. 63/876,975 filed Sep. 6, 2025 and 63/908,051 filed Oct. 30, 2025, both of which are fully incorporated herein by reference.

The present application also claims priority to U.S. Provisional Patent Application Ser. No. 63/877,017 filed Sep. 6, 2025, which is fully incorporated herein by reference.

### TECHNICAL FIELD

The present disclosure is directed to prosthetic valve replacement system, particularly to devices, systems, and methods for transcatheter delivery of expandable prosthetic valves, more particularly to a delivery handle arrangement for an expandable prosthetic valve, and even more particularly to delivery system that includes a delivery handle that has a flex adjustment mechanism that facilitates in delivery of a medical device to a treatment site, and still even more particularly to a delivery system that includes a delivery handle that has a flex adjustment mechanism, and wherein the delivery system can be used for delivery of a medical device (e.g., expandable prosthetic heart valve, stent, LAA (left atrial appendage) closure device, etc.), and wherein the delivery system includes a catheter that includes a flexible tube, spring, braid, etc. to facilitate in the controlled flexing of the distal region of the flexible catheter.

### BACKGROUND OF DISCLOSURE

Many cardiovascular devices such as expandable heart valves are inserted into a patient via the patient's vascular system and then expanded at the treatment site. These devices are typically crimped onto catheter prior to insertion into a patient. Medical devices such as transcatheter aortic valves (TAVs) represent a significant advancement in prosthetic valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; 12,383,399 and US Pub. No. 2020/0405482, all of which are incorporated fully herein by reference.

A TAV is designed to be compressed into a small diameter catheter, remotely placed within a patient's diseased aortic valve to take over the function of the native valve. Some TAVs are balloon-expandable, while others are self-expandable. In both cases, the TAVs are deployed within a calcified native valve that is forced permanently open and becomes the surface against which the stent is held in place by friction. TAVs can also be used to replace failing bioprosthetic or transcatheter valves, commonly known as a valve in valve procedure. Major TAVR advantages to the traditional surgical approaches include refraining cardiopulmonary bypass and/or aortic cross-clamping and sternotomy that significantly reduces patients' morbidity.

However, several complications are associated with current TAV devices, such as mispositioning, crimp-induced leaflet damage, paravalvular leak, thrombosis, conduction abnormalities, and prosthesis-patient mismatch. These complications are potentially associated with the calcification landscape of the native valve, geometric and mechanical properties of the aortic root, blood biochemistry and coagulability associated with the patient, and concomitant conditions such as hypertension, coronary artery disease, heart failure, etc.

TAVR involves delivery, deployment, and implantation of a crimped, stented valve within a diseased aortic valve or degenerated bioprosthesis. Prosthetic valves that are collapsible can be delivered into a patient via a tube-like delivery apparatus such as a catheter, a trocar, a laparoscopic instrument, or the like, thus avoiding more invasive procedures such as full open-chest, open-heart surgery.

When the collapsed prosthetic valve has reached the desired treatment site in the patient (e.g., patient's heart valve that is to be replaced by the prosthetic valve), the prosthetic valve can be expanded in the treatment site.

In conventional delivery systems for prosthetic heart valves, the delivery system needs to navigate through the vascular system to deliver the prosthetic heart valve to a treatment site. The vascular system includes winding paths that can be difficult to traverse and delivery a medical device to the treatment site without damaging the medical device, the delivery system and/or the walls and/or tissue of the vascular system.

There therefore is a need for further improvements to the devices, systems, and methods for transcatheter delivery of collapsible prosthetic heart valves.

### SUMMARY OF DISCLOSURE

The present disclosure is directed to medical device delivery arrangements, particularly to devices, systems, and methods for delivering medical devices to a treatment site, more particularly to delivery system that includes a delivery handle that has a flex adjustment mechanism that facilitates in delivery of a medical device to a treatment site, and even more particularly to a delivery system that includes a delivery handle that has a flex adjustment mechanism, and wherein the delivery system can be used for delivery of a medical device (e.g., stent, TAV, heart valve, LAA (left atrial appendage) closure device, etc.), and wherein the delivery system includes a catheter that includes a flexible tube, spring, braid, etc. to facilitate in the controlled flexing of the distal region of the flexible catheter.

In one non-limiting aspect of the present disclosure, the delivery handle arrangement includes a flex adjustment mechanism configured to bend or flex the distal region of the flexible catheter or to bend or flex the distal region of the balloon catheter at a certain angle and/or orientation. Such flex adjustment arrangement can be used to a) properly position and/or orient a medical device at a treatment site (e.g., center medical device at the treatment site), and/or b) navigate the medical device and/or distal region of the balloon catheter and/or flexible catheter about bends in a vascular system when moving the medical device in the vascular system and towards the treatment site.

In another and/or alternative aspect of the present disclosure, there is provided a delivery system that includes a delivery handle, wherein the delivery handle includes a housing that has a flex adjustment mechanism that includes a flex knob or flex adjustor. In one non-limiting embodiment, flex knob or flex adjustor is movably connected to the housing of the delivery handle (e.g., rotatable connected to the housing of the delivery handle, slidably connected to the housing of the delivery handle, etc.). In another and/or alternative embodiment, the flex knob or flex adjustor can include a body portion (e.g., threaded body portion, etc.) that is partially or fully positioned in the body cavity of the housing. The flex knob or flex adjustor can be coupled (e.g., threadedly coupled, slidably coupled, etc.) to a flex insert and/or flex actuator (e.g., threaded flex insert and/or flex actuator, etc.), and wherein the flex insert and/or flex actuator is partially or fully contained within the housing, and wherein the flex knob or flex adjustor is configured to cause the flex insert and/or flex actuator to move (e.g., rotate, slide, etc.) within the housing during movement of the flex knob or flex adjustor. In one non-limiting configuration, the flex knob or flex adjustor is configured to engage a flex insert and/or flex actuator (e.g., threaded flex insert, threaded flex actuator, etc.) and to cause the flex insert and/or flex actuator to move during movement of the flex knob or flex adjustor. In another non-limiting configuration, the flex insert and/or flex actuator is configured to move within the housing (e.g., move along a longitudinal axis of the housing, etc.) during movement of the flex knob or flex adjustor. In another non-limiting configuration, the flex insert and/or flex actuator is connected to one end of a wire member, and which wire member is connected at an opposite end to the flexible catheter. The type of connection used to connect the wire member to the flexible catheter and to the flex insert and/or flex actuator is non-limiting (e.g., clamp, weld, friction connection, adhesive, melted connection, etc.). In another non-limiting embodiment, rotation of the flex knob or flex adjustor in a first direction causes the flex insert and/or flex actuator to translate linearly along the longitudinal axis of the housing away from the distal end of the housing towards the proximal end of the housing, and wherein the wire member pulls the distal end or distal region of the flexible catheter or the balloon catheter to cause bending or flexing or increased bending or flexing of the distal region of the flexible catheter or the balloon catheter at a certain angle and/or orientation, and rotation of the flex knob or flex adjustor in a second opposite direction causes the flex insert and/or flex actuator to translate linearly along the longitudinal axis of the housing away from the proximal end of the housing towards the distal end of the housing, and wherein the wire member unpulls the distal end or distal region of the flexible catheter or the balloon catheter to cause less bending or less flexing or no bending or no flexing of the distal region of the flexible catheter or the balloon catheter.

In another and/or alternative aspect of the present disclosure, there is provided a delivery system includes a delivery handle, and wherein the delivery handle includes a housing having an inner core that extends linearly between a proximal end and a distal end of the housing, and wherein the inner core is connected and/or receives a portion of a flexible catheter and optionally an expandable balloon catheter that is disposed coaxially with the flexible catheter; and wherein the delivery handle includes a flex adjustment mechanism that is configured to cause controlled bending or flexing of the flexible catheter and/or balloon catheter at a certain angle at a distal region of the flexible catheter and/or balloon catheter. In one non-limiting embodiment, the flex adjustment mechanism includes a flex knob or flex adjustor that is positioned on the housing of the delivery handle. In another non-limiting embodiment, the flex knob or flex adjustor is configured to be moved by a user (e.g., flex knob or flex adjustor can be rotated by a user, flex knob or flex adjustor can be slid on the housing by a user, etc.) to enable the user to controllably bend or flex a distal region of the flexible catheter and/or balloon catheter. In one non-limiting configuration, the flex knob or flex adjustor optionally includes a body portion that is partially or fully located in within the housing (e.g., partially or located within the core of the housing, etc.); and wherein the body portion of the flex knob or flex adjustor is configured to engage a flex insert and/or flex actuator that is partially or fully located within the housing; and wherein the flex insert and/or flex actuator is configured to engage a portion of the body portion and to be caused to move when the flex knob or flex adjustor is rotated. In another non-limiting configuration, the flex knob or flex adjustor optionally includes a threaded body portion that is partially or fully located in within the housing (e.g., partially or located within the core of the housing, etc.); and wherein the threaded body of the flex knob or flex adjustor is configured to threadedly engage a flex insert and/or flex actuator; and wherein at least a portion of the threaded body of the flex knob or flex adjustor and the flex insert and/or flex actuator is positioned within the housing; and wherein movement (e.g., rotation, sliding, etc.) of the flex knob or flex adjustor causes the flex insert and/or flex actuator to move either toward or away from a distal end of the housing; and wherein the flex insert and/or flex actuator is connected to wire member (e.g., pull-wire); and wherein the wire member is positioned in at least a portion of the flexible catheter; and wherein the wire member is moveable relative to an outer surface of the flexible catheter; and wherein movement of the flex insert and/or flex actuator causes the wire member to move relative to the outer surface of the flexible catheter; and wherein movement of the wire member causes a distal region of the flexible catheter and/or the balloon catheter to controllably bend or flex and/or unbend. In another non-limiting embodiment, the flex knob or flex adjustor is optionally located at the distal region of the housing (e.g., the flex knob or flex adjustor is positioned at or spaced from the distal end of the housing a distance of 0.1-15% [and all values and ranges therebetween] of the longitudinal length of the housing). In another non-limiting configuration, the flex knob or flex adjustor is rotatably connected to the outer surface of the housing such that the flex knob or flex adjustor rotates about a central longitudinal axis of the housing. In another non-limiting configuration, the flex insert and/or flex actuator that is located partially or fully within the housing is configured to not rotate within the housing, but to only move along a longitudinal axis of the housing. In another non-limiting configuration, the average width of the flex knob or flex adjustor is 0.1-40% (and all values and ranges therebetween) of the longitudinal length of the housing. In another non-limiting embodiment, the flex knob or flex adjustor includes one or more grip members (e.g., grooves, rough surface, slots, ribs, etc.) to facilitate in the gripping and/or moving of the flex knob or flex adjustor.

In another and/or alternative non-limiting aspect of the present disclosure, the delivery handle includes a flex adjustment mechanism that is configured to cause controlled bending of the flexible catheter at a certain angle at a distal region of the flexible catheter and/or balloon catheter; and wherein the flex adjustment mechanism includes a flex knob or flex adjustor that includes a body portion; and wherein the body portion is partially or fully contained within the housing of the delivery handle; and wherein there is a flex insert and/or flex actuator that is partially or fully positioned within the housing and is configured to engage with the body of the flex knob or flex adjustor; and wherein movement of the flex knob or flex adjustor causes the flex insert and/or flex actuator to move either toward or away from a distal end of the housing; and wherein the distal region of the flexible catheter includes a flexible hypotube, spring and/or flexible tube and a pull ring; and wherein one end of a wire member is coupled to the pull ring and the other end of the wire member is connected to the flex insert and/or flex actuator; and wherein the wire member is positioned in at least a portion of the flexible catheter; and wherein the wire member is moveable relative to an outer surface of the flexible catheter; and wherein movement of the flex insert and/or flex actuator causes the wire member to move relative to the outer surface of the flexible catheter; and wherein movement of the wire member causes a distal region of the flexible catheter and/or balloon catheter to controllably bend or flex and/or unbend. In one non-limiting embodiment, the flex adjustment mechanism includes a flex knob or flex adjustor that includes a threaded body portion; and wherein the threaded body portion is partially or fully contained within the housing; and wherein there is a flex insert and/or flex actuator that is partially or fully positioned within the housing and is configured to threadedly engage with the threaded body of the flex knob or flex adjustor; and wherein rotation or movement of the flex knob or flex adjustor causes the flex insert and/or flex actuator to translate linearly along the housing; and wherein one end of a wire member is coupled to a pull ring located in a distal region of the flexible catheter and the other end of the wire member is connected to the flex insert and/or flex actuator; and wherein the wire member is positioned in at least a portion of the flexible catheter; and wherein the wire member is moveable relative to an outer surface of the flexible catheter; and wherein movement of the flex insert and/or flex actuator toward a proximal end of the housing of the delivery handle causes the wire member to pull the pull ring toward the delivery handle to thereby cause the distal region that includes the hypotube, spring and/or flexible tube in the distal region of the flexible catheter to bend or flex; and wherein movement of the flex insert and/or flex actuator toward a distal end of the housing of the delivery handle causes the wire member to unpull the pull ring to thereby cause the distal region that includes the hypotube, spring and/or flexible tube in the distal region of the flexible catheter to unbend or unflex. In another non-limiting embodiment, the hypotube, spring and/or flexible tube in the distal region of the flexible catheter is configured to a) enable the hypotube, spring and/or flexible tube to bend when the flex insert and/or flex actuator causes the wire member to pull the pull ring toward the proximal end of the delivery handle, and/or b) cause the distal region of the flexible catheter to partially or fully restraighten to a linear or near linear shape as the wire member allows the pull ring to move away from the delivery handle when the flex insert and/or flex actuator moves toward the distal end of the housing. The configuration of the hypotube, spring and/or flexible tube is non-limiting. The material used to form of the hypotube, spring and/or flexible tube is non-limiting. In another non-limiting embodiment, the hypotube, spring and/or flexible tube is formed of a shape memory material. In another non-limiting embodiment, the hypotube, spring and/or flexible tube is formed of a rhenium and/or hafnium alloy. In another non-limiting embodiment, the hypotube, spring and/or flexible tube is formed of a polymer material.

In another and/or alternative non-limiting aspect of the present disclosure, the delivery handle optionally includes a flex adjustment mechanism that has a flex indicator to indicate a degree/amount of flex at the distal region of the flexible catheter. In one non-limiting embodiment, the flex indicator includes one or more of a needle indicator, digital/electronic indicator, visual indicator, color indicator, sound indicator, bar indicator, light indicator, etc. to provide information to the user of the degree and/or amount of bending or flexing of the distal region of the flexible catheter and/or balloon catheter. In one non-limiting configuration, the flex indicator includes a needle indicator, digital indicator, or bar indicator that is configured to move as the flex insert and/or flex actuator and/or other component of the delivery handle moves relative to the housing so as to indicate to a user a degree of flex or bend that is present at the distal region of the flexible catheter and/or balloon catheter. The flex indicator can optionally include numbers, letters, bars, sloped graphics, shapes, or other markings to provide the user additional information on the degree or amount of flex or bend in the flexible catheter and/or balloon catheter. In another non-limiting configuration, the flex adjustment mechanism includes a flex knob or flex adjustor that includes a body portion; and wherein the body portion is partially or fully contained within the housing; and wherein there is a flex insert and/or flex actuator that is configured to engaged with the body of the flex knob or flex adjustor; and wherein the flex insert and/or flex actuator includes a flex indicator (e.g., needle, etc.); and wherein rotation of the flex knob or flex adjustor causes the flex insert and/or flex actuator to move either toward or away from a distal end of the housing; and wherein movement of the flex insert and/or flex actuator causes the flex indicator to move to indicate a degree of bending and/or flexing of the distal region of the flexible catheter and/or balloon catheter. In one non-limiting configuration, the outer portion of the housing of the delivery handle can optionally include one or more indicator windows. In another non-limiting configuration, the one or more indicator windows can include a partially or fully transparent material covering (e.g., clear plastic, clear glass, etc.) to enable a user to see through the partially or fully transparent material covering and into indicator window to view of the flex indicator. In another non-limiting configuration, the housing includes two indicator windows locate at or near the distal end of the housing and wherein the indicator windows are optionally located directly opposite one another on the housing. In another non-limiting configuration, the longitudinal length of each of the indicator windows is generally 0.1-10% (and all values and ranges therebetween) of the longitudinal length of the housing. In another non-limiting configuration, the maximum width of the indicator window is 0.1-40% (and all values and ranges therebetween) of the maximum perimeter of the housing. In another non-limiting configuration, the one or more indicator windows are located rearwardly of the flex knob or flex adjustor.

In another and/or alternative non-limiting aspect of the present disclosure, the expandable medical device that is used with the delivery system is a prosthetic heart valve.

In another and/or alternative non-limiting aspect of the present disclosure, the delivery handle of the delivery system includes a flex adjustment mechanism that is configured to provide significantly more flexion of the distal region of a flexible catheter and/or balloon catheter as compared to current state of the art delivery arrangements. The delivery system in accordance with the present disclosure can be used to deliver a medical device to a heart such as, but not limited to, a TAV. Transcatheter Aortic Valve Replacement (TAVR) involves delivering a prosthetic aortic valve endovascularly to the native aortic annulus using a delivery system. For an expandable prosthetic valve, the delivery system is optionally includes a balloon catheter for use with an expandable prosthetic valve which is used to expand the prosthetic valve, a flexible catheter which is used to guide the expandable valve through the vascular system and a handle which serves as the user interface for the delivery system. When inserting a TAV in a patient, typically access to the patient's arterial system is via the common femoral artery and the delivery system is advanced through the descending aorta, around the aortic arch to the aortic annulus. The aortic arch has significant curvature to it which requires the delivery system to navigate across without damaging the vessel. Additionally, the aortic root can often be very angled (>70°) making delivery of the valve challenging. To navigate around the aortic arch, and to aid in positioning and moving the prosthetic valve coaxially within the aortic root, it is advantageous to use a delivery system that can be flexed on the distal region of the delivery system, such as moving the distal region of the flexible catheter in one direction in a curved shape. Flexing of the flexible catheter during advancement around the aortic arch can be used to reduce system insertional forces, thereby minimizing arterial wall stress and mitigating risk of vascular injury. Once the delivery system has reached the aortic annulus, the ability to being able to flex the flexible catheter at such location can aid in ensuring the prosthetic valve is positioned coaxial to the aortic root, thereby improving prosthetic valve delivery at the treatment site. In accordance with one non-limiting configuration of the present disclosure, controlled flexion of the distal region of the flexible catheter can be accomplished by placing a pull ring within the distal region of the flexible catheter, which flexible catheter has a wire member (e.g., pull-wire, etc.) connected at one end to the pull ring. The wire member can optionally be partially or fully encased within a protective tube (e.g., PTFE tube, etc.) and runs along a portion or all of the length of the flexible catheter, and optionally within the wall thickness of the flexible catheter. The distal region of the flexible catheter can optionally be constructed using a braid material (e.g., metallic braid, etc.), a hypotube (e.g., a laser cut hypotube, etc.), an optional flexible tube, etc. to provide structural support and/or to facilitate in flexion of the distal region of the flexible catheter. When causing the distal region of the flexible catheter to controllably bend or flex, the wire member is pulled by one or more components of the handle of the delivery arrangement toward the proximal end of the housing, which in turn causes a tensile force to be applied to the pull ring, which in turn causes the distal region of the flexible catheter to deflect, flex or bend in a desired direction. The direction of the flex or bend can be controlled by a) the connection location of the wire member on the pull ring, b) the location of the wire member on the flexible catheter and/or balloon catheter, and/or c) the design and configuration of the spring, hypotube, braid and/or flex tube in the distal region of the flexible catheter. The flex adjustment mechanism in accordance with the present disclosure can be configured to enable the flexible catheter to be flexed or bent significantly more than the maximum flex or bending of prior art delivery devices.

In another and/or alternative non-limiting aspect of the present disclosure the flexible catheter can include a) an optional inner liner (e.g., PTFE liner, etc.), b) an optional outer layer (e.g., Pebax™ inner layer, etc.) that is positioned over the inner liner, c) an optional braid reinforcement that is position along a portion or all of the longitudinal length of the flexible catheter (e.g., 51-100% the length of the flexible catheter and all values and ranges therebetween) and is optionally positioned about the optional inner and outer liner, d) an a hypotube (e.g., a laser cut hypotube, etc.), spring, and/or flexible tube that is positioned on the distal region of the flexible catheter (e.g., hypotube, spring and/or flexible tube has a longitudinal length of 1-20 cm [and all values and ranges therebetween] and is positioned 0-10 cm [and all values and ranges therebetween] of the distal end of the flexible catheter), e) a wire member that is positioned 80-100% (and all values and ranges therebetween) of the longitudinal length of the flexible catheter, f) a pull ring that is positioned at or near the distal end (e.g., 0-10 cm and all values and ranges therebetween of the distal end) of i) the hypotube, spring, and/or flexible tube or b) the distal end of the flexible catheter, and wherein the wire member is connected (e.g., weld, clamp, solder, twist connection, adhesive, melted connection, etc.) to the pull ring, g) an outer layer of protective material (e.g., Pebax™, etc.) that forms the outer layer of the flexible catheter, which outer layer is positioned over the construction of the flexible catheter so that the components of the flexible catheter properly move together to create the flexible catheter, and wherein the optional braid reinforcement or the outer layer of protective material includes a wire cavity for the wire member to be positioned therein and moveable therein, and wherein one or more other tubes, guide wires, fluid passageways are positioned within the cavity of the inner liner and/or outer liner. In another non-limiting configuration, there is optionally a cavity liner in the wire cavity (e.g., PTFE liner, etc.) that extends 10-100% (and all values and ranges therebetween) the longitudinal length of the wire cavity. In one non-limiting configuration, the cross-sectional area of the wire cavity is greater than a cross-sectional area of the wire member. In another non-limiting configuration, the shape and size of the pull ring is non-limiting. In another non-limiting configuration, the width of the pull rings is 0.001-2 cm (and all values and ranges therebetween).

In another and/or alternative non-limiting aspect of the present disclosure, the delivery handle of the delivery system includes a flex adjustment mechanism that is configured to provide significantly more flexion of the distal region of a flexible catheter as compared to current state of the art delivery arrangements, and wherein the delivery handle is configured to accommodate a longer linear pull of the wire member, which then causes the distal region of the flexible catheter to have an improve range of flex or bending.

In another and/or alternative non-limiting aspect of the present disclosure, the delivery handle facilitates the linear movement of the wire member, wherein the wire member is attached at one end to the flex insert and/or flex actuator by crimping a crimp band, and wherein the crimp band is attached to the flex insert and/or flex actuator. In one non-limiting embodiment, the delivery handle is configured such as the flex knob or flex adjustor is turned, the flex insert and/or flex actuator move within the housing, thereby applying tension or releasing tension on the wire member, and wherein the flex insert and/or flex actuator travel along the outer surface of the body core. In operation, by increasing the linear length of the threaded portion of the flex knob or flex adjustor, and the length of the body core of the housing, additional length of travel for the flex insert and/or flex actuator allows for a longer pull of the wire member toward the proximal end of the housing, thereby increasing the degree of flex or bend that is achieved on the distal region of the flexible catheter. In one non-limiting configuration, the flex adjustment mechanism in accordance with the present disclosure is able to achieve maximum flexing of the flexible catheter of over 180°, and up to 360° (e.g., maximum flex 181-360° and all values and ranges therebetween).

In another and/or alternative non-limiting aspect of the present disclosure, the medical devices that can be used with the delivery system in accordance with the present disclosure include, but are not limited to, stent, prosthetic heart valve, TAVR valve, mitral valve replacement, tricuspid valve replacement, pulmonary valve replacement, LAA (left atrial appendage) closure device, etc., In one non-limiting implementation, the delivery system is configured to deliver a prosthetic valve to a treatment site (e.g., in a patient's heart valve, etc.). More particularly, example implementations of the disclosed technology provide a delivery system that is configured to more accurately deliver such prosthetic valves to the treatment site. The delivery handle of the delivery system in accordance with the present disclosure includes: (i) a flex adjustment mechanism that is configured to facilitate the bending and/or flexing of the flexible catheter and/or balloon catheter to facilitate in the movement of the medical device and a portion of the flexible catheter and/or balloon catheter about around various bends or arches (e.g., aortic arch bend) during the delivery of the medical device to a treatment site.

In another and/or alternative non-limiting aspect of the present disclosure, the prosthetic valve includes a radially collapsible and expandable frame and a leaflet structure comprising a plurality of leaflets. The prosthetic valve can optionally include an outer skirt disposed on at least a portion of an outer surface of the expandable frame and overlying a plurality of cells of at least a portion of the frame. The prosthetic valve can also or alternatively include an inner skirt. The expandable frame can optionally comprise a plurality of interconnected struts and strut joints defining a plurality of open cells in the expandable frame. As can be appreciated, the expandable frame can have other configurations. The expandable frame is optionally partially (e.g., 1-99.999 wt. % and all values and ranges therebetween) or fully made of a metal material.

In another and/or alternative non-limiting aspect of the present disclosure, the medical device is in the form of a prosthetic valve that includes a frame, a leaflet structure supported by the frame, and an optional inner skirt secured to the surface of the frame and/or leaflet structure, and an optional outer skirt that is secured to the frame. The prosthetic valve can be implanted in the annulus of the native aortic valve; however, the prosthetic valve also can be configured to be implanted in other valves of the heart (e.g., tricuspid valve, pulmonary valve, mitral valve). The prosthetic valve has a “lower” end and an “upper” end, wherein the lower end of the prosthetic valve is the inflow end and the upper end of the prosthetic valve is the outflow end. The metal alloy that is optionally used to partially or fully form the expandable frame of the prosthetic valve is configured to be radially collapsible to a collapsed or crimped state for introduction into the body (e.g., on a delivery catheter, balloon catheter, etc.) and radially expandable to an expanded state for implanting the prosthetic valve at a desired location in the body (e.g., the aortic valve, tricuspid valve, pulmonary valve, mitral valve, etc.). The expandable frame of the prosthetic valve can be formed of a plastically-expandable material that permits crimping of the expandable frame to a smaller profile for delivery and expansion of the expandable frame. The expansion of the crimped expandable frame of the prosthetic valve can be by an expansion device such as, but not limited to, a balloon of on a balloon catheter; however, the expandable frame can optionally be partially (e.g., 1-99.999 wt. % and all values and ranges therebetween) or fully formed of a self-expanding material (e.g., Nitinol, etc.). The expandable frame is optionally at least partially (e.g., 1-99.999 wt. % and all values and ranges therebetween) formed of a plurality of angularly spaced, vertically extending posts, or struts. The posts or struts can optionally be interconnected via a lower row of circumferentially extending struts and an upper row of circumferentially extending struts via strut joints. The struts can be arrangement in a variety of patterns (e.g., zig-zag pattern, saw-tooth pattern, triangular pattern, polygonal pattern, oval pattern, etc.). One or more of the posts and/or struts can have the same or different thicknesses and/or cross-sectional shape and/or cross-sectional area.

In another and/or alternative non-limiting aspect of the present disclosure, the inner skirt of the prosthetic valve can be formed of a variety of flexible materials (e.g., polymer [e.g., polyethylene terephthalate (PET), polyester, nylon, Kevlar,®, silicon, etc.], composite material, metal, fabric material, etc.). In one non-limiting embodiment, the material used to partially (e.g., 1-99.999 wt. % and all values and ranges therebetween) or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The size, configuration, and thickness of the inner skirt is non-limiting (e.g., thickness of 0.1-20 mils and all values and ranges therebetween). The inner skirt can be secured to the inside and/or outside of the frame using various means (e.g., sutures, clamp arrangement, etc.). The inner skirt can be connected to one or more of the leaflets of the leaflet arrangement.

In another and/or alternative non-limiting aspect of the present disclosure, the outer skirt of the prosthetic valve can be used to 1) at least partially seal and/or prevent paravalvular leakage. The outer skirt can be positioned at least partially (e.g., 1-99.999 wt. % and all values and ranges therebetween) about the exterior region of the frame. The outer skirt or sleeve generally is positioned completely around a portion of the outside of the frame. Generally, the outer skirt is positioned about the lower portion of the frame, but does not fully cover the upper half of the frame; however, this is not required. The outer skirt can be connected to the frame by a variety of arrangements (e.g., sutures, adhesive, melted connection, clamping arrangement, etc.). At least a portion of the outer skirt can optionally be located on the interior surface of the frame. Generally, the outer skirt is formed of a more flexible and/or compressible material than the inner skirt; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material to provide additional structural integrity to the outer skirt. The size, configuration, and thickness of the outer skirt is non-limiting. The thickness of the outer skirt is generally 0.1-20 mils (and all values and ranges therebetween).

In another and/or alternative non-limiting aspect of the present disclosure, the prosthetic valve can include a leaflet structure that can be attached to the expandable frame and/or outer and/or inner skirts. The connection arrangement used to secure the leaflet structure to the expandable frame and/or inner and/or outer skirts is non-limiting (e.g., sutures, staples, melted bold, adhesive, clamp arrangement, etc.). The material used to form the leaflet structure can include bovine pericardial tissue, biocompatible synthetic materials, or various other suitable natural or synthetic materials. The leaflet structure generally comprises two or more leaflets (e.g., 2, 3, 4, 5, 6, etc.). In one non-limiting arrangement, the leaflet structure includes three leaflets arranged to collapse in a tricuspid arrangement. The configuration of the leaflet structures is non-limiting. The leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, etc.). The leaflets can optionally include reinforcing structures or strips to 1) facilitate in securing the leaflets together, 2) facilitate in securing the leaflets to the skirt and/or frame, and/or 3) inhibit or prevent tearing or other types of damage to the leaflets.

In another and/or alternative non-limiting aspect of the present disclosure, the expandable frame of the medical device can be partially (e.g. 1-99.999 wt. % and all values and ranges therebetween) or fully formed of a metal alloy that includes a) stainless-steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy (e.g., stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween), or n) metal alloy (e.g., stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) hafnium (e.g., 5-99 awt. % hafnium and all values and ranges therebetween). As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or 5-15 awt. % hafnium in the metal alloy is referred to as the “rhenium effect” or “hafnium effect.” As defined herein, a “rhenium effect” or a “hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc. As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents.

In another and/or alternative non-limiting aspect of the present disclosure, the metal alloy used to partially or fully form the expandable frame of the medical device can include a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, l) refractory metal alloy, m) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 atomic weight percent (awt. %) or atomic percent (awt. %) rhenium (e.g., 5-99 awt. % rhenium and all values and ranges therebetween), or n) metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 atomic weight percent (awt. %) or atomic percent (awt. %) hafnium (e.g., 5-99 awt. % hafnium and all values and ranges therebetween). It has been found that for several metal alloys the inclusion of rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or 5-15 awt. % hafnium in the metal alloy is referred to as the “rhenium effect” or “hafnium effect.” As defined herein, a “rhenium effect” or a “hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy.

In another and/or alternative non-limiting aspect of the present disclosure, the metal alloy used to partially or fully form the expandable frame of the medical device can include stainless-steel, CoCr alloys, TiAlV alloys, aluminum alloys, nickel alloys, titanium alloys, tungsten alloys, molybdenum alloys, copper alloys, MP35N alloys, or beryllium-copper alloys that have been modified to include at least 5-15 awt. % rhenium and/or 5-15 awt % hafnium so as to result in improved ductility and/or tensile strength as compared to the same metal alloy that is absent rhenium and/or hafnium. As defined herein, a stainless-steel alloy (SS alloy) includes at least 50 wt. % iron (e.g., 50-85 wt. % and all values and ranges therebetween), 10-30 wt. % chromium, 0-35 wt. % nickel, and optionally one or more of 0-5 wt. % molybdenum, 0-6 wt. % manganese, 0-1 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-72 wt. % cobalt, 15-35 wt. % chromium, and optionally one or more of 1-38 wt. % nickel, 2-18 wt. % molybdenum, 0-18 wt. % iron, 0-1 wt. % titanium, 0-2.8 wt. % manganese, 0-0.15 wt. % silver, 0-2 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox™ and Elgiloy™ alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium. As defined herein, a stainless-steel alloy (SS alloy) includes at least 50 wt. % iron (e.g., 50-85 wt. % and all values and ranges therebetween), 10-30 wt. % chromium, 0-35 wt. % nickel, and optionally one or more of 0-5 wt. % molybdenum, 0-6 wt. % manganese, 0-1 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-72 wt. % cobalt, 15-35 wt. % chromium, and optionally one or more of 1-38 wt. % nickel, 2-18 wt. % molybdenum, 0-18 wt. % iron, 0-1 wt. % titanium, 0-2.8 wt. % manganese, 0-0.15 wt. % silver, 0-2 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox™ and Elgiloy™ alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium. It will be appreciated that all component ranges of the alloys set forth in this application include the range, the end points of the range, and all values and ranges therebetween.

In another and/or alternative non-limiting aspect of the present disclosure, the metal alloy used to partially or fully form the expandable frame of the medical device can include at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium effect. In one non-limiting embodiment, the metal alloy is a stainless-steel alloy that has been modified to include at least 5-15 awt. % rhenium. In another and/or alternative non-limiting embodiment, the metal alloy is a cobalt-chromium alloy that has been modified to include at least 5-15 awt. % rhenium. In another and/or alternative non-limiting embodiment, the metal alloy is a TiAlV alloy that has been modified to include at least 5-15 awt. % rhenium. In another and/or alternative non-limiting embodiment, the metal alloy is an aluminum alloy that has been modified to include at least 5-15 awt. % rhenium. In another and/or alternative non-limiting embodiment, the metal alloy is a nickel alloy that has been modified to include at least 5-15 awt. % rhenium. In another and/or alternative non-limiting embodiment, the metal alloy is a titanium alloy that has been modified to include at least 5-15 awt. % rhenium. In another and/or alternative non-limiting embodiment, the metal alloy is a tungsten alloy that has been modified to include at least 15 awt. % rhenium. In another and/or alternative non-limiting embodiment, the metal alloy is a molybdenum alloy that has been modified to include at least 5-15 awt. % rhenium. In another and/or alternative non-limiting embodiment, the metal alloy is a copper alloy that has been modified to include at least 5-15 awt. % rhenium. In another and/or alternative non-limiting embodiment, the metal alloy is a beryllium-copper alloy that has been modified to include at least 5-15 awt. % rhenium.

The metal alloy used to partially or fully form the expandable frame of the medical device can include at least 5 awt. % (e.g., 5-99 awt. % and all values and ranges therebetween) hafnium, and 0.1-96 wt. % (and all values and ranges therebetween) of one or more additives selected from the group of aluminum, boron, beryllium, bismuth, cadmium, calcium, cerium, chromium, cobalt, copper, gallium, gold, iridium, iron, lanthanum, lithium, magnesium, manganese, molybdenum, nickel, niobium, osmium, palladium, platinum, rare earth metals, rhenium, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a hafnium effect. In one non-limiting embodiment, the metal alloy is a stainless-steel alloy that has been modified to include at least 5-15 awt. % hafnium. In another and/or alternative non-limiting embodiment, the metal alloy is a cobalt chromium alloy that has been modified to include at least 5-15 awt. % hafnium. In another and/or alternative non-limiting embodiment, the metal alloy is a TiAlV alloy that has been modified to include at least 5-15 awt. % hafnium. In another and/or alternative non-limiting embodiment, the metal alloy is an aluminum alloy that has been modified to include at least 5-15 awt. % hafnium. In another and/or alternative non-limiting embodiment, the metal alloy is a nickel alloy that has been modified to include at least 5-15 awt. % hafnium. In another and/or alternative non-limiting embodiment, the metal alloy is a titanium alloy that has been modified to include at least 5-15 awt. % hafnium. In another and/or alternative non-limiting embodiment, the metal alloy is a tungsten alloy that has been modified to include at least 5-15 awt. % hafnium. In another and/or alternative non-limiting embodiment, the metal alloy is a molybdenum alloy that has been modified to include at least 5-15 awt. % hafnium. In another and/or alternative non-limiting embodiment, the metal alloy is a copper alloy that has been modified to include at least 5-15 awt. % hafnium. In another and/or alternative non-limiting embodiment, the metal alloy is a beryllium-copper alloy that has been modified to include at least 5-15 awt. % hafnium.

In another and/or alternative non-limiting aspect of the present disclosure, the metal alloy used to partially or fully form the expandable frame of the medical device can include rhenium and/or hafnium, and molybdenum, and the weight percent of rhenium and/or hafnium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other components other than the additives (e.g., carbon, oxygen, phosphorous, sulfur, hydrogen, lead, nitrogen, etc.). In one non-limiting embodiment, the metal alloy is fully formed of or includes rhenium and/or hafnium, and molybdenum, and the weight percent of rhenium and/or hafnium plus the combined weight percent of additives is greater than the weight percent of molybdenum, and the metal alloy optionally includes 0-2 wt. % of a combination of other components other than the additives (e.g., carbon, oxygen, phosphorous, sulfur, hydrogen, lead, nitrogen, etc.).

In another and/or alternative non-limiting aspect of the present disclosure, the metal alloy used to partially or fully form the expandable frame of the medical device can include rhenium and/or hafnium, and molybdenum, and one or more additives selected from bismuth, niobium, tantalum, tungsten, titanium, vanadium, chromium, manganese, yttrium, zirconium, technetium, ruthenium, rhodium, hafnium, osmium, copper, and iridium, and the atomic weight percent of rhenium and/or hafnium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In another and/or alternative non-limiting aspect of the present disclosure, the metal alloy used to partially or fully form the expandable frame of the medical device can include at least 5 awt. % (e.g., 5-99 awt. % and all values and ranges therebetween) rhenium and/or hafnium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another and/or alternative non-limiting embodiment, the metal alloy includes rhenium and/or hafnium, molybdenum, and chromium. In another and/or alternative non-limiting embodiment, the metal alloy includes at least 35 wt. % (e.g., 35-75 wt. % and all values and ranges therebetween) rhenium and/or hafnium, and the metal alloy also includes chromium. In one non-limiting embodiment, the metal alloy includes at least 35 wt. % rhenium and/or hafnium, and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another and/or alternative non-limiting embodiment, the metal alloy includes 15-50 awt. % rhenium and/or hafnium (and all values and ranges therebetween) and 0.5-70 awt. % chromium (and all values and ranges therebetween). In another and/or alternative non-limiting embodiment, the metal alloy includes 15-50 awt. % rhenium and/or hafnium (and all values and ranges therebetween) and 0.5-70 awt. % tantalum (and all values and ranges therebetween). In another and/or alternative non-limiting embodiment, the metal alloy includes 15-50 awt. % rhenium and/or hafnium (and all values and ranges therebetween) and 0.5-70 awt. % niobium (and all values and ranges therebetween). In another and/or alternative non-limiting embodiment, the metal alloy includes 15-50 awt. % rhenium and/or hafnium (and all values and ranges therebetween) and 0.5-70 awt. % titanium (and all values and ranges therebetween). In another and/or alternative non-limiting embodiment, the metal alloy includes 15-50 awt. % rhenium and/or hafnium (and all values and ranges therebetween) and 0.5-70 awt. % zirconium (and all values and ranges therebetween). In another and/or alternative non-limiting embodiment, the metal alloy includes 15-50 awt. % rhenium and/or hafnium (and all values and ranges therebetween) and 0.5-70 awt. % molybdenum (and all values and ranges therebetween). In another and/or alternative non-limiting embodiment, the metal alloy includes at least 15 awt. % rhenium and/or hafnium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In another and/or alternative non-limiting aspect of the present disclosure, the metal alloy used to partially or fully form the expandable frame of the medical device can include a refractory metal alloy, and wherein the refractory metal alloy includes at least 20 wt. % of one or more of niobium, rhenium, tantalum, molybdenum or tungsten (e.g., 20-99.9 wt. % and all values and ranges therebetween), and 0.1-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In another and/or alternative non-limiting aspect of the present disclosure, the metal alloy used to partially or fully form the expandable frame of the medical device can include a refractory metal alloy, and wherein the refractory metal alloy includes at least 5 awt. % rhenium and/or hafnium, at least 20 wt. % of one or more of niobium, tantalum, molybdenum or tungsten (e.g., 20-99.9 wt. % and all values and ranges therebetween), and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In another and/or alternative non-limiting aspect of the present disclosure, the metal alloy used to partially or fully form the expandable frame of the medical device can include a refractory metal alloy, and wherein the refractory metal alloy includes at least 20 wt. % of one or more of niobium, tantalum or tungsten, and wherein the refractory metal alloy includes 0-30 wt. % molybdenum (and all values and ranges therebetween), and wherein the refractory metal alloy includes at least 5 awt. % rhenium and/or hafnium (e.g., 5-80 awt. % rhenium and/or hafnium and all values and ranges therebetween), and wherein the refractory metal alloy includes and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In another and/or alternative non-limiting aspect of the present disclosure, the metal alloy used to partially or fully form the expandable frame of the medical device can include at least 5 awt. % rhenium and/or hafnium (e.g., 5-99 awt. % rhenium and/or hafnium and all values and ranges therebetween), and at least 0.1 wt. % of one or more additive metals selected from aluminum, bismuth, chromium, cobalt, copper, hafnium, iridium, iron, magnesium, manganese, nickel, niobium, osmium, rhodium, ruthenium, silicon, silver, tantalum, technetium, titanium, tungsten, vanadium, yttrium, and zirconium, and wherein the metal alloy includes 0-30 wt. % molybdenum (and all values and ranges therebetween), and wherein a combined weight percent of rhenium and/or hafnium, and the additive metals is 70-100 wt. % (and all values and ranges therebetween).

In another and/or alternative non-limiting aspect of the present disclosure, the metal alloy used to partially or fully form the expandable frame of the medical device can include stainless-steel that has been modified with at least 5 awt. % rhenium and/or hafnium (e.g., 5-50 awt. % rhenium and/or hafnium and all values and ranges therebetween), and wherein a combined weight percent of iron, chromium, nickel, tantalum, niobium, copper, manganese, aluminum, titanium, selenium, vanadium, tungsten, hafnium and rhenium is 70-100 wt. % (and all values and ranges therebetween).

In another and/or alternative non-limiting aspect of the present disclosure, the metal alloy used to partially or fully form the expandable frame of the medical device can include cobalt-chromium alloy that has been modified with at least 5 awt. % rhenium and/or hafnium (e.g., 5-50 awt. % rhenium and/or hafnium and all values and ranges therebetween), and wherein a combined weight percent of cobalt, chromium, nickel, iron, titanium, manganese, silver, tungsten, silicon, aluminum, iron, boron, silver, titanium, hafnium, and rhenium is 70-100 wt. % (and all values and ranges therebetween).

In another and/or alternative non-limiting aspect of the present disclosure, the metal alloy used to partially or fully form the expandable frame of the medical device can include titanium-aluminum-vanadium alloy that has been modified with at least 5 awt. % rhenium and/or hafnium (e.g., 5-50 awt. % rhenium and/or hafnium and all values and ranges therebetween), and wherein a combined weight percent of aluminum, vanadium, titanium, iron, yttrium, hafnium, and rhenium is 70-100 wt. % (and all values and ranges therebetween).

In another and/or alternative non-limiting aspect of the present disclosure, the metal alloy used to partially or fully form the expandable frame of the medical device can include aluminum alloy that has been modified with at least 5 awt. % rhenium and/or hafnium (e.g., 5-50 awt. % rhenium and/or hafnium and all values and ranges therebetween), and wherein a combined weight percent of aluminum, silicon, magnesium, manganese, scandium, beryllium, yttrium, cerium, chromium, iron, zinc, titanium, lithium, silver, calcium, zirconium, cadmium, bismuth, nickel, vanadium, gallium, copper, hafnium, and rhenium is 70-100 wt. % (and all values and ranges therebetween).

In another and/or alternative non-limiting aspect of the present disclosure, the metal alloy used to partially or fully form the expandable frame of the medical device can include nickel alloy that has been modified with at least 5 awt. % rhenium and/or hafnium (e.g., 5-50 awt. % rhenium and/or hafnium and all values and ranges therebetween), and wherein a combined weight percent of nickel, chromium, iron, copper, cobalt, aluminum, tantalum, tungsten, titanium, niobium, silicon, hafnium, and rhenium is 70-100 wt. % (and all values and ranges therebetween).

In another and/or alternative non-limiting aspect of the present disclosure, the metal alloy used to partially or fully form the expandable frame of the medical device can include titanium alloy that has been modified with at least 5 awt. % rhenium and/or hafnium (e.g., 5-50 awt. % rhenium and/or hafnium and all values and ranges therebetween), and wherein a combined weight percent of titanium, aluminum, tin, palladium, vanadium, nickel, ruthenium, chromium, zirconium, niobium, silicon, cobalt, iron, hafnium, and rhenium is 70-100 wt. % (and all values and ranges therebetween).

In another and/or alternative non-limiting aspect of the present disclosure, the metal alloy used to partially or fully form the expandable frame of the medical device can include tungsten alloy that has been modified with at least 5 awt. % rhenium and/or hafnium (e.g., 5-50 awt. % rhenium and/or hafnium and all values and ranges therebetween), and wherein a combined weight percent of tungsten, nickel, copper, iron, hafnium, and rhenium is 70-100 wt. % (and all values and ranges therebetween).

In another and/or alternative non-limiting aspect of the present disclosure, the metal alloy used to partially or fully form the expandable frame of the medical device can include copper alloy that has been modified with at least 5 awt. % rhenium and/or hafnium (e.g., 5-50 awt. % rhenium and/or hafnium and all values and ranges therebetween), and wherein a combined weight percent of copper, zinc, tin, iron, silicon, manganese, aluminum, beryllium, cobalt, nickel, hafnium, and rhenium is 70-100 wt. % (and all values and ranges therebetween). In another and/or alternative non-limiting aspect of the present disclosure.

In another and/or alternative non-limiting aspect of the present disclosure, the metal alloy used to partially or fully form the expandable frame of the medical device can include beryllium-copper alloy that has been modified with at least 5 awt. % rhenium and/or hafnium (e.g., 5-50 awt. % rhenium and/or hafnium and all values and ranges therebetween), and wherein a combined weight percent of copper, beryllium, cobalt, silicon, hafnium, and rhenium is 70-100 wt. % (and all values and ranges therebetween).

In another and/or alternative non-limiting aspect of the present disclosure, the metal alloy used to partially or fully form the expandable frame of the medical device can include titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In another and/or alternative non-limiting aspect of the present disclosure, the metal alloy used to partially or fully form the expandable frame of the medical device can includes a primary metal (5-95 wt. % primary metal and all values and ranges therebetween) selected from one or more of molybdenum, rhenium, hafnium, niobium, tantalum, tungsten, and one or more alloying agents such as, but are not limited to, calcium, carbon, cerium oxide, chromium, cobalt, copper, gold, hafnium, iron, lanthanum oxide, magnesium, nickel, osmium, platinum, rare earth metals, rhenium, silver, technetium, titanium, vanadium, yttrium, yttrium oxide, zinc, zirconium, zirconium oxide, and/or alloys of one or more of such components (e.g., MoHfC, MoY₂O₃, MoCs₂O, MoW, MoTa, MoZrO₂, MoLa₂O₃, MoRe alloy, MoReW alloy, HfMo alloy, HfW alloy, ReW alloy, etc.), and wherein metal alloy optionally includes less than about 5 wt. % (e.g., 0-4.999999 wt. % and all values and ranges therebetween) of a combination a) metals of than the primary metal, b) metals or compounds other the alloying agents, c) oxygen, d) nitrogen, and e) other metals and/or impurities.

In another and/or alternative non-limiting aspect of the present disclosure, the metal alloy used to partially or fully form the expandable frame of the medical device can include tungsten and copper and optionally one or more metal agents such as, but are not limited to, calcium, carbon, cerium oxide, chromium, cobalt, gold, hafnium, iron, lanthanum oxide, magnesium, molybdenum, nickel, niobium, osmium, platinum, rare earth metals, rhenium, silver, tantalum, technetium, titanium, vanadium, yttrium, yttrium oxide, zinc, zirconium, zirconium oxide, and/or alloys of one or more of such components. In one non-limiting formulation, the metal alloy includes 1-99.9 wt. % tungsten (and all values and ranges therebetween) (e.g., 1 wt. %, 1.01 wt. %, 1.02 wt. % . . . 99.88 wt. %, 99.89 wt. %, 99.9 wt. %), and 0.1-99 wt. % copper (and all values and ranges therebetween) (e.g., 0.1 wt. %, 0.101 wt. %, 0.102 wt. % . . . 98.998 wt. %, 98.999 wt. %, 99 wt. %). In another non-limiting formulation, the tungsten constitutes the greatest weight percent in the metal alloy and the copper constitutes the second greatest weight percent in the metal alloy. In another non-limiting formulation, the tungsten constitutes the largest weight percent of any component that forms the metal alloy. In another non-limiting formulation, the tungsten constitutes greater than 50 wt. % of the metal alloy.

In another and/or alternative non-limiting aspect of the present disclosure, the metal alloy used to partially or fully form the expandable frame of the medical device can include tungsten and rhenium and/or hafnium, and optionally one or more alloying agents such as, but not limited to, calcium, carbon, cerium oxide, chromium, cobalt, copper, gold, hafnium, iron, lanthanum oxide, magnesium, molybdenum, nickel, niobium, osmium, platinum, rare earth metals, rhenium, silver, tantalum, technetium, titanium, tungsten, vanadium, yttrium, yttrium oxide, zinc, zirconium, zirconium oxide, and/or alloys of one or more of such components (e.g., WRe, WReMo, WHf, WHfMo, WHfRe, WHfReMo, etc.). In one non-limiting formulation, the metal alloy includes 1-40 wt. % rhenium and/or hafnium (and all values and ranges therebetween and 60-99 wt. % tungsten (and all values and ranges therebetween). The total weight percent of the tungsten, rhenium and hafnium in the metal alloy is at least about 95 wt. % (e.g., 95-100% and all values and ranges therebetween). In another non-limiting formulation, the metal alloy includes 1-47.5 wt. % rhenium and/or hafnium (and all values and ranges therebetween) and 20-80 wt. % tungsten (and all values and ranges therebetween) and 0-47.5 wt. % molybdenum (and all values and ranges therebetween). The total weight percent of the tungsten, molybdenum, rhenium and hafnium in the metal alloy is at least about 95 wt. % (e.g., 95-100% and all values and ranges therebetween). In one non-limiting specific metal alloy, the weight percent of the tungsten is greater than a weight percent of rhenium, hafnium and/or molybdenum. In another non-limiting specific metal alloy, the weight percent of the tungsten is greater than 50 wt. % of the metal alloy. In another non-limiting specific metal alloy, the weight percent of the tungsten is greater than a weight percent of rhenium and/or hafnium, but is less than a weigh percent of molybdenum. In another non-limiting specific metal alloy, the weight percent of the tungsten is greater than a weight percent of molybdenum, but less than a weigh percent of rhenium and/or hafnium. In another non-limiting metal alloy, the weight percent of the tungsten is less than a weight percent of rhenium, hafnium and/or molybdenum.

In another and/or alternative non-limiting aspect of the present disclosure, the metal alloy used to partially or fully form the expandable frame of the medical device can include a metal alloy that has an atomic weight percent of rhenium and/or hafnium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium in the metal alloy is 0.7:1 to 1.5:1 (and all values and ranges therebetween).

In another and/or alternative non-limiting aspect of the present disclosure, the metal alloy used to partially or fully form the expandable frame of the medical device can include two of bismuth, niobium, tantalum, tungsten, titanium, vanadium, chromium, manganese, yttrium, zirconium, technetium, ruthenium, rhodium osmium, copper, and iridium, the atomic ratio of the two metals is 0.4:1 to 2.5:1 (and all values and ranges therebetween).

In another and/or alternative non-limiting aspect of the present disclosure, the metal alloy used to partially or fully form the expandable frame of the medical device can include a titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In another and/or alternative non-limiting aspect of the present disclosure, the metal alloy used to partially or fully form the expandable frame of the medical device can include at least 15 awt. % hafnium and/or rhenium and also includes one or more of molybdenum, niobium, tantalum, or tungsten.

In another and/or alternative non-limiting aspect of the present disclosure, the metal alloy used to partially or fully form the expandable frame of the medical device can include at least 15 awt. % hafnium and/or rhenium and 0.1-75 wt. % (and all values and ranges therebetween) of one or more of aluminum, bismuth, calcium, carbon, chromium, cobalt, copper, gold, iridium, iron, lanthanum, magnesium, manganese, molybdenum, nickel, niobium, osmium, platinum, rare earth metals, rhodium, ruthenium, silver, tantalum, technetium, titanium, tungsten, vanadium, yttrium, zinc, zirconium, and/or alloys of one or more of such components.

In another and/or alternative aspect of the present disclosure, the metal alloy used to partially or fully form the expandable frame of the medical device includes at least 15 awt. % hafnium and/or rhenium, nickel and tungsten and optionally one or more alloying agents such as, but not limited to, aluminum, bismuth, calcium, carbon, chromium, cobalt, copper, gold, iron, magnesium, molybdenum, niobium, osmium, platinum, rare earth metals, rhenium, silver, tantalum, technetium, titanium, vanadium, yttrium, zinc, zirconium, and/or alloys of one or more of such components (e.g., WNi, WNiMo, WNiRe, etc.). In another non-limiting formulation, the metal alloy that includes at least 15 awt. % hafnium and/or rhenium includes 1-47.5 wt. % nickel (and all values and ranges therebetween) and 20-80 wt. % tungsten (and all values and ranges therebetween) and 1-47.5 wt. % (and all values and ranges therebetween) of one or more of aluminum, bismuth, calcium, carbon, chromium, cobalt, copper, gold, hafnium, iron, magnesium, molybdenum, nickel, niobium, osmium, platinum, rare earth metals, silver, tantalum, technetium, titanium, vanadium, yttrium, zinc, zirconium.

In another and/or alternative non-limiting aspect of the present disclosure, the metal alloy used to partially or fully form the expandable frame of the medical device can include at least 15 awt. % hafnium and/or rhenium and nickel.

In another and/or alternative non-limiting aspect of the present disclosure, the metal alloy used to partially or fully form the expandable frame of the medical device can include at least 15 awt. % hafnium and/or rhenium includes (e.g., 15-99 awt. % and all values and ranges therebetween), optionally 5 awt. % (e.g., 5-99 awt. % and all values and ranges therebetween) molybdenum, optionally 5 awt. % (e.g., 5-99 awt. % and all values and ranges therebetween) niobium, optionally 5 awt. % (e.g., 5-99 awt. % and all values and ranges therebetween) tantalum, optionally 5 awt. % (e.g., 5-99 awt. % and all values and ranges therebetween) tungsten, and at least 0.1 wt. % (e.g., 0.1 wt. % to 96 wt. % and all values and ranges therebetween) of one or more of aluminum, boron, beryllium, bismuth, cadmium, calcium, cerium, chromium, cobalt, copper, gallium, gold, iridium, iron, lanthanum, lithium, magnesium, manganese, nickel, osmium, palladium, platinum, rare earth metals, rhodium, ruthenium, scandium, silver, silicon, technetium, tin, titanium, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen.

In another and/or alternative non-limiting aspect of the present disclosure, the expandable frame can be formed of at least 30 wt. % (e.g., 30-100 wt. % and all values and ranges therebetween) of the metal alloy.

In another and/or alternative non-limiting aspect of the present disclosure, the metal alloy used to partially or fully form the expandable frame of the medical device can include less than 5 wt. % nickel (e.g., 0-4.99 wt. % and all values and ranges therebetween).

In another and/or alternative non-limiting aspect of the present disclosure, the metal alloy used to partially or fully form the expandable frame of the medical device can include less than 5 wt. % chromium (e.g., 0-4.99 wt. % and all values and ranges therebetween).

In another and/or alternative non-limiting aspect of the present disclosure, the metal alloy used to partially or fully form the expandable frame of the medical device is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printing, 3D printed coatings, chemical vapor deposition, chemical polishing, swaging, cleaning, pickling, ion beam deposition or implantation, sputter coating, vacuum deposition, nitriding, laser cutting, EDM cutting, gun-drilling, compression, sintering, compression process, consolidation process, etc.

In another and/or alternative non-limiting aspect of the present disclosure, the metal alloy used to partially or fully form the expandable frame of the medical device optionally has a generally uniform density throughout the metal alloy, and also results in the desired yield and ultimate tensile strengths of the metal alloy. In one non-limiting embodiment, the density of the metal alloy that includes at least 15 awt. % rhenium and/or hafnium and/or is a refractory metal alloy is generally at least about 5 μm/cc (e.g., 5 μm/cc-21 μm/cc and all values and ranges therebetween; 10-20 μm/cc; etc.), and typically at least about 11-19 μm/cc.

In another and/or alternative non-limiting aspect of the present disclosure, the metal alloy used to partially or fully form the expandable frame of the medical device optionally includes a certain amount of carbon and oxygen; however, this is not required. The carbon to oxygen atomic ratio can be as low as about 0.2:1 (e.g., 0.2:1 to 50:1 and all values and ranges therebetween).

In another and/or alternative non-limiting aspect of the present disclosure, the metal alloy optionally includes a controlled amount of nitrogen; however, this is not required. In one non-limiting formulation, the metal alloy includes less than about 0.001 wt. % nitrogen (e.g., 0 wt. % to 0.0009999 wt. % and all values and ranges therebetween). In one non-limiting formulation of the metal alloy, the atomic ratio of carbon to nitrogen ion the metal alloy is at least about 1.5:1 (e.g., 1.5:1 to 400:1 and all values and ranges therebetween). In another non-limiting formulation of the metal alloy, the atomic ratio of oxygen to nitrogen is at least about 1.2:1 (e.g., 1.2:1 to 150:1 and all value and ranges therebetween).

In another and/or alternative non-limiting aspect of the present disclosure, the metal alloy used to partially or fully form the expandable frame of the medical device optionally is at least partially or fully formed from a tube or rod of metal alloy, or is formed into a shape that is at least 80% of the final net shape of the medical device (e.g., formed by 3D printing, formed by compression and/or sintering of metal alloy powder, formed by gun-drilling and/or EDM cutting, formed by swaging, etc.). When the metal alloy is formed into a rod, the rod can optionally be gun-drilled and/or subjected to EDM cutting or otherwise cut to form a tube from the rod.

In another and/or alternative non-limiting aspect of the present disclosure, the metal alloy used to partially or fully form the expandable frame of the medical device optionally can be at least partially or fully formed from by 3D printing. As can be appreciated, other portions of the medical device or other type of device can be formed by 3D printing.

In another and/or alternative non-limiting aspect of the present disclosure, the metal alloy used to partially or fully form the expandable frame of the medical device is optionally subjected to a swaging process; however, this is not required.

In another and/or alternative non-limiting aspect of the present disclosure, the metal alloy used to partially or fully form the expandable frame of the medical device can optionally be nitrided; however, this is not required.

In another and/or alternative non-limiting aspect of the present disclosure, the metal alloy used to partially or fully form the expandable frame of the medical device, just prior to or after being partially or fully formed into the desired medical device or other type of device, can optionally be cleaned, polished, sterilized, nitrided, etc., for final processing of the metal alloy.

In another and/or alternative non-limiting aspect of the present disclosure, the metal alloy or medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. The one or more agents can be coated on and/or impregnated in medical device by a variety of mechanisms such as, but not limited to, spraying (e.g., atomizing spray techniques, etc.), powder deposition, dip coating, flow coating, dip-spin coating, roll coating (direct and reverse), sonication, brushing, plasma deposition, depositing by vapor deposition, MEMS technology, and rotating mold deposition.

In another and/or alternative non-limiting aspect of the present disclosure, the one or more agents on and/or in the metal alloy or medical device, when used on the medical device, can be released in a controlled manner so the area in question to be treated is provided with the desired dosage of agent over a sustained period of time. One or more polymers can be used to at least partially control the release of one or more agents from the metal alloy or medical device can be porous or non-porous. The one or more agents can be inserted into and/or applied to one or more surface structures and/or micro-structures on the metal alloy or medical device, and/or be used to at least partially form one or more surface structures and/or micro-structures on the metal alloy or medical device.

In another and/or alternative non-limiting aspect of the present disclosure, the medical device can optionally include a marker material that facilitates enabling the medical device to be properly positioned in a body passageway (e.g., blood vessel, heart valve, etc.). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque).

In another and/or alternative non-limiting aspect of the present disclosure, one or more components of the prosthetic valve (e.g., frame, inner skirt, outer skirt, leaflets, material used to secure leaflets to frame, etc.) can be partially (e.g., 1% to 99.99% and all values and ranges therebetween) or fully be coated with an enhancement coating to improve one or more properties of the prosthetic heat valve (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement coating forms a surface that is less of an irritant to cell about the coated surface after the prosthetic valve is implanted, reduces the rate to which cells grown on coated surface after prosthetic valve is implanted, reduce rate to which leaflets fail to properly operate after prosthetic valve is implanted, promote production and/or release of NO, etc.). In one non-limiting embodiment, only the expandable frame of the prosthetic valve includes the enhancement coating, and wherein the frame is partially (e.g., 1-99.99% and all values and ranges therebetween) or fully coated with the enhancement coating. In another non-limiting embodiment, only one or more of all of the leaflets of the prosthetic valve include the enhancement coating, and wherein one or more or all of the leaflets are partially (e.g., 1-99.99% and all values and ranges therebetween) or fully coated with the enhancement coating. In another non-limiting embodiment, only the inner skirt of the prosthetic valve includes the enhancement coating, and wherein the inner skirt is partially (e.g., 1-99.99% and all values and ranges therebetween) or fully coated with the enhancement coating. In another non-limiting embodiment, only the outer skirt of the prosthetic valve includes the enhancement coating, and wherein the outer skirt is partially (e.g., 1-99.99% and all values and ranges therebetween) or fully coated with the enhancement coating. In another non-limiting embodiment, two or more or all of a) the expandable frame, b) one or more or all of the leaflets, c) the inner skirt and d) the outer skirt of the prosthetic valve are partially (e.g., 1-99.99% and all values and ranges therebetween) or are fully coated with the enhancement coating. Non-limiting enhancement coatings that can be applied to a portion or all of the outer surface of one or more components of the prosthetic valve includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZnNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. In one non-limiting embodiment, the one or more enhancement coatings are optionally applied to a portion or all of the outer surface of one or more components of the prosthetic valve by a vacuum process using an energy source to vaporize material and deposit a thin layer of enhancement coating material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In another non-limiting embodiment, the thickness of the enhancement coating is greater than 1 nanometer (e.g., 2 nanometers to 100 microns and all values and ranges therebetween), and typically 0.1-25 microns, and more typically 0.2-10 microns. As defined herein, the enhancement coating does not include and is different from the optional agent coating or the optional nitriding coating.

One non-limiting object of the present disclosure is the provision of a delivery system in accordance with the present disclosure that facilitates the delivery of an expandable medical device to a treatment site.

In another and/or alternative non-limiting object of the present disclosure is the provision of a delivery handle that includes a) a housing that has an inner core that extends linearly between a proximal end and a distal end of the housing, and wherein the inner core at least partially receives a flexible catheter and/or balloon catheter, and b) a flex adjustment mechanism that is configured to bend or flex the distal region flexible catheter or balloon catheter at a certain angle.

In another and/or alternative non-limiting object of the present disclosure is the provision of a delivery handle that includes a flex adjustment mechanism that is configured to bend or flex the distal region of a flexible catheter and/or a balloon catheter at a certain angle, and wherein the flex adjustment mechanism includes a) a flex knob or flex adjustor that has or is coupled to a body (e.g., a threaded body, etc.), wherein the body is at least partially contained within the housing of the delivery handle, b) a flex insert and/or flex actuator (e.g., a threaded flex insert and/or flex actuator, etc.) that is engages with the body and is caused by the body to linearly move within the housing during movement of the flex knob or flex adjustor, and c) a wire member that coupled (e.g., clamped, welded, adhesively connected, melted connection, etc.) at one end to the flex insert and/or flex actuator and/or other component and the other end of the wire member is connected to a distal region of the flexible catheter or balloon catheter; and wherein movement of the wire member causes flexing or bend, or unflexing or unbending of the distal region of the flexible catheter or balloon catheter.

In another and/or alternative non-limiting object of the present disclosure is the provision of a delivery handle that optionally includes a flex indicator that is used to indicate the amount and/or a degree of flex or bend present in the distal region of the flexible catheter and/or balloon catheter, and wherein the flex indicator that optionally includes a needle and/or other visual indicator (e.g., meter, digital readout, electronic readout, bend or flex symbol, number, percentage value, bend angle value, etc.) that is used to indicate the amount and/or a degree of flex or bend present in the flexible catheter and/or balloon catheter.

In another and/or alternative non-limiting object of the present disclosure is the provision of a delivery handle that is used with a prosthetic valve, stent, LAA (left atrial appendage) closure device, etc.

In another and/or alternative non-limiting object of the present disclosure is the provision of a system that delivers an expandable medical device to a treatment site, and wherein the system includes a flex adjustment mechanism that is configured to bend or flex a distal region of the flexible catheter and/or balloon catheter at certain angle, and wherein the flex adjustment mechanism includes i) a flex knob or flex adjustor that is movably (e.g., rotatably, etc.) coupled to a housing of the delivery handle, and wherein the flex knob or flex adjustor includes a body (e.g., a threaded body, etc.), and wherein the body is at least partially contained within the housing, ii) a flex insert and/or flex actuator (e.g., a threaded flex insert and/or flex actuator, etc.) that is engaged with the body, and wherein movement of the flex knob or flex adjustor causes the flex insert and/or flex actuator to move linearly along a longitudinal axis of the housing, and c) a wire member that at one end is coupled to the distal region of the flexible catheter and/or balloon catheter (e.g., connected via one or more of a crimp band, weld or solder connection, adhesive connection, clamp or other mechanical connection, melted connection, etc. at the distal region of the flexible catheter or flexible catheter) and at the other end of the wire member to the flex insert and/or flex actuator and/or other component of the flex adjustment mechanism (e.g., connected to the flex insert and/or flex actuator by a crimp band, weld or solder connection, clamp connection or other mechanical connection, adhesive connection, melted connection, etc.), and wherein rotation, sliding or other type of movement of the flex knob or flex adjustor causes the flex insert and/or flex actuator and/or other component of the flex adjustment mechanism to translate linearly along the longitudinal axis of the housing of the delivery handle, and wherein the wire member pulls/pushes the distal region of the flexible catheter and/or balloon flexible catheter to cause bending/unbending of the distal region of the flexible catheter and/or balloon catheter.

In another and/or alternative non-limiting object of the present disclosure is the provision of a system that delivers an expandable medical device to a treatment site, and wherein the system includes a flex indicator and flex indicator window, wherein the flex indicator is configured to move within the flex indicator window to indicate the degree, amount, angle, etc. of flex or bend of the distal region of the flexible catheter and/or balloon catheter.

In another and/or alternative non-limiting object of the present disclosure is the provision of a delivery handle arrangement that facilitates a delivery of an expandable medical device to a treatment site; and the delivery handle arrangement comprising a) a housing having an inner core that at least partially extends linearly between a proximal end and a distal end of the housing; b) a flexible catheter that is connected to the housing; c) a flex adjustment mechanism that is configured to controllably bend the flexible catheter; and the flex adjustment mechanism includes i) a flex knob or flex adjustor that is movably connected to the housing; the flex knob or flex adjustor includes a body portion that is at least partially positioned in the inner core of the housing; ii) a flex insert and/or flex actuator that is at least partially positioned in the inner core of the housing; the flex insert and/or flex actuator is in engagement with the body portion; the flex insert and/or flex actuator is caused to move by movement of the flex knob or flex adjustor; and iii) a wire member; the wire member is coupled at one end to the flex insert and/or flex actuator and at the other end to a distal region of the flexible catheter; and wherein the distal region of the flexible catheter is caused to controllably bend during movement of the flex knob or flex adjustor.

In another and/or alternative non-limiting object of the present disclosure is the provision of a delivery handle arrangement that facilitates a delivery of an expandable medical device to a treatment site, wherein the flex knob or flex adjustor is configured to rotate on the housing; and the flex knob or flex adjustor is configured to cause a portion of the wire member to be drawn into the housing when rotated in a first direction, and wherein the distal region of the flexible catheter is caused to bend when the flex knob or flex adjustor is rotated in the first direction; and the flex knob or flex adjustor is configured to cause the wire member to be moved outwardly from the housing when rotated in a second direction, and wherein the distal region of the flexible catheter is caused to unbend when the flex knob or flex adjustor is rotated in the second direction.

In another and/or alternative non-limiting object of the present disclosure is the provision of a delivery handle arrangement that facilitates a delivery of an expandable medical device to a treatment site, and further includes a first flex indicator arrangement that is located on the housing; and the first flex indicator arrangement includes a first flex indicator that is configured to provide information to a user about a degree or amount of bending or flexing of the distal region of the flexible catheter prior, during and/or after the flex knob or flex adjustor is moved.

In another and/or alternative non-limiting object of the present disclosure is the provision of a delivery handle arrangement that facilitates a delivery of an expandable medical device to a treatment site, and wherein the first flex indicator arrangement includes a flex indicator window on an outer surface of the housing and wherein the first flex indicator is covered by the flex indicator window.

In another and/or alternative non-limiting object of the present disclosure is the provision of a delivery handle arrangement that facilitates a delivery of an expandable medical device to a treatment site, and wherein the first flex indicator includes a movable needle and a flex bend graphic; and wherein the needle translates along and/or move relative to the housing and along or about a portion of the flex bend graphic to indicate to a user a degree or amount of bending or flexing of the distal region of the flexible catheter.

In another and/or alternative non-limiting object of the present disclosure is the provision of a delivery handle arrangement that facilitates a delivery of an expandable medical device to a treatment site, and wherein the first flex indicator is located rearwardly of the flex knob or flex adjustor.

In another and/or alternative non-limiting object of the present disclosure is the provision of a delivery handle arrangement that facilitates a delivery of an expandable medical device to a treatment site, and further including a second flex indicator arrangement; and wherein the second flex indicator arrangement includes a second flex indicator; and wherein the second flex indicator is spaced from the first flex indicator (e.g., positioned on an opposite side of the housing from the first flex indicator, etc.).

In another and/or alternative non-limiting object of the present disclosure is the provision of a delivery handle arrangement that facilitates a delivery of an expandable medical device to a treatment site, and wherein the flex knob or flex adjustor does not move along a longitudinal axis of the housing where the flex knob or flex adjustor is moved on the housing.

In another and/or alternative non-limiting object of the present disclosure is the provision of a delivery handle arrangement that facilitates a delivery of an expandable medical device to a treatment site, and wherein the distal end of the flexible catheter includes a pull ring; and wherein the wire member is connected to the pull ring; and wherein the pull ring is positioned at or spaced from a distal end of the flexible catheter.

In another and/or alternative non-limiting object of the present disclosure is the provision of a delivery handle arrangement that facilitates a delivery of an expandable medical device to a treatment site, and wherein the distal region of the flexible catheter includes a flexible hypotube or spring; the flexible hypotube or spring is configured to a) bend when the distal region of the flexible catheter is caused to bend by the flex adjustment mechanism, and b) cause the distal region of the flexible catheter to unbend when the flex adjustment mechanism allows the distal region of the flexible catheter to unbend from a bent position the flexible hypotube or spring is positioned further from the distal end of the flexible catheter than the pull ring.

In another and/or alternative non-limiting object of the present disclosure is the provision of a delivery handle arrangement that facilitates a delivery of an expandable medical device to a treatment site, and wherein the distal region of the flexible catheter includes the flexible hypotube; and wherein the flexible hypotube is optionally a laser cut hypotube that is optionally formed of a shape memory material.

In another and/or alternative non-limiting object of the present disclosure is the provision of a delivery handle arrangement that facilitates a delivery of an expandable medical device to a treatment site, wherein a distal region of the flexible catheter includes an inner layer of material that includes a wire cavity; and wherein the wire cavity is configured to receive a portion of the wire member; and wherein the wire member is configured to move within the wire cavity.

In another and/or alternative non-limiting object of the present disclosure is the provision of a delivery handle arrangement that facilitates a delivery of an expandable medical device to a treatment site, and wherein the inner layer of material in the flexible catheter is a braided material layer.

In another and/or alternative non-limiting object of the present disclosure is the provision of a delivery handle arrangement that facilitates a delivery of a medical device to a treatment site; the delivery handle arrangement comprising a) a housing having an inner core that at least partially extends linearly between a proximal end and a distal end of the housing; b) a flexible catheter that is connected to the housing; and c) a flex adjustment mechanism that is configured to controllably bend a distal region the flexible catheter; the flex adjustment mechanism includes: i) a flex knob that is movably connected to the housing; the flex knob includes a body portion that is at least partially positioned in the inner core of the housing; ii) a flex insert and/or flex actuator that is at least partially positioned in the inner core of the housing; the flex insert and/or flex actuator is configured to move within the housing during movement of the flex knob; and iii) a wire member; the wire member is coupled at a proximal end to the flex insert and/or flex actuator and a distal region of the flexible catheter; and wherein the distal region of the flexible catheter is caused to controllably bend during movement of the flex knob.

In another and/or alternative non-limiting object of the present disclosure is the provision of a delivery handle arrangement wherein the flex knob is configured to rotate on the housing; the flex knob is configured to cause a portion of the wire member to be drawn into the housing when rotated in a first direction, and wherein the distal region of the flexible catheter is caused to bend when the flex knob is rotated in the first direction; the flex knob is configured to cause the wire member to be moved outwardly from the housing when rotated in a second direction, and wherein the distal region of the flexible catheter is caused to unbend when the flex knob is rotated in the second direction.

In another and/or alternative non-limiting object of the present disclosure is the provision of a delivery handle arrangement further including a first flex indicator arrangement located on the housing; the first flex indicator arrangement includes a first flex indicator that is configured to provide information to a user about a degree or amount of bending of the distal region of the flexible catheter prior, during and/or after the flex knob is rotated.

In another and/or alternative non-limiting object of the present disclosure is the provision of a delivery handle arrangement wherein the first flex indicator arrangement includes a flex indicator window on an outer surface of the housing, and wherein the a first flex indicator is covered by the flex indicator window.

In another and/or alternative non-limiting object of the present disclosure is the provision of a delivery handle arrangement wherein the first flex indicator includes a movable needle and a flex bend graphic; and wherein the needle moves relative to the flex bend graphic to indicate to a user a degree of bending of the distal region of the flexible catheter.

In another and/or alternative non-limiting object of the present disclosure is the provision of a delivery handle arrangement wherein the first flex indicator is located rearwardly of the flex knob.

In another and/or alternative non-limiting object of the present disclosure is the provision of a delivery handle arrangement further including a second flex indicator arrangement; the second flex indicator arrangement includes a second flex indicator; the second flex indicator is positioned on an opposite side of the housing from the first flex indicator.

In another and/or alternative non-limiting object of the present disclosure is the provision of a delivery handle arrangement wherein the flex knob does not move along a longitudinal axis of the housing when the flex knob is moved on the housing.

In another and/or alternative non-limiting object of the present disclosure is the provision of a delivery handle arrangement wherein the distal end of the flexible catheter includes a pull ring; the wire member is connected to the pull ring; the pull ring is spaced from a distal end of the flexible catheter.

In another and/or alternative non-limiting object of the present disclosure is the provision of a delivery handle arrangement wherein a longitudinal axis of the wire member when connected to the pull ring is parallel to a longitudinal axis of a central cavity of the pull ring; the wire member is connected to an outer surface of the pull ring.

In another and/or alternative non-limiting object of the present disclosure is the provision of a delivery handle arrangement wherein the distal region of the flexible catheter includes a flexible hypotube or spring; the flexible hypotube or spring is configured to a) bend when the distal region of the flexible catheter is caused to bend by the flex adjustment mechanism, and b) unbend when the flex adjustment mechanism allows the distal region of the flexible catheter to unbend from a bent position by the flex adjustment mechanism; the flexible hypotube or spring is positioned further from the distal end of the flexible catheter than the pull ring.

In another and/or alternative non-limiting object of the present disclosure is the provision of a delivery handle arrangement wherein the pull ring is connected to a distal end of the flexible hypotube or spring.

In another and/or alternative non-limiting object of the present disclosure is the provision of a delivery handle arrangement wherein the distal region of the flexible catheter includes the flexible hypotube; the flexible hypotube is a laser cut hypotube.

In another and/or alternative non-limiting object of the present disclosure is the provision of a delivery handle arrangement wherein the wire member is positioned in a cavity of the flexible hypotube or spring and runs under a top surface of the flexible hypotube or spring along a longitudinal length of the flexible hypotube or spring.

In another and/or alternative non-limiting object of the present disclosure is the provision of a delivery handle arrangement wherein a distal region of the flexible catheter includes an inner layer of material that includes a wire cavity; the wire cavity is configured to receive a portion of the wire member; the wire member is configured to move within the wire cavity.

In another and/or alternative non-limiting object of the present disclosure is the provision of a delivery handle arrangement wherein the inner layer of material is a braided material layer that includes the wire cavity.

In another and/or alternative non-limiting object of the present disclosure is the provision of a delivery handle arrangement wherein the inner layer of material is positioned rearwardly of flexible hypotube or spring that is located in a distal region of the flexible catheter.

In another and/or alternative non-limiting object of the present disclosure is the provision of a delivery handle arrangement In another and/or alternative non-limiting object of the present disclosure is the provision of a delivery handle arrangement wherein the expandable medical device is a prosthetic heart valve.

In another and/or alternative non-limiting object of the present disclosure is the provision of a system that delivers an expandable medical device to a treatment site, the system comprising: a) a flexible catheter; and b) a delivery handle arrangement; the delivery handle arrangement comprising: i) a housing having an inner core that at least partially extends linearly between a proximal end and a distal end of the housing; and ii) a flex adjustment mechanism that is configured to controllably bend a distal region the flexible catheter; the flex adjustment mechanism includes: A) a flex knob that is movably connected to the housing; the flex knob includes a body portion that is at least partially positioned in the inner core of the housing; B) a flex insert and/or flex actuator that is at least partially positioned in the inner core of the housing; the flex insert and/or flex actuator is configured to move within the housing during movement of the flex knob; and C) a wire member; the wire member is coupled at a proximal end to the flex insert and/or flex actuator and a distal region of the flexible catheter; and wherein the flexible catheter that is connected to the housing; and wherein the distal region of the flexible catheter is caused to controllably bend during movement of the flex knob; and wherein movement of the flex knob in a first direction causes the flex insert and/or flex actuator to translate linearly along a longitudinal axis of the housing to cause movement of the wire member to cause the flexible catheter to controllably bend the flexible catheter.

In another and/or alternative non-limiting object of the present disclosure is the provision of a system that delivers an expandable medical device to a treatment site wherein the flex knob is configured to rotate on the housing; the flex knob is configured to cause a portion of the wire member to be drawn into the housing when rotated in a first direction, and wherein the distal region of the flexible catheter is caused to bend when the flex knob is rotated in the first direction; the flex knob is configured to cause the wire member to be moved outwardly from the housing when rotated in a second direction, and wherein the distal region of the flexible catheter is caused to unbend when the flex knob is rotated in the second direction.

In another and/or alternative non-limiting object of the present disclosure is the provision of a system that delivers an expandable medical device to a treatment site further including a first flex indicator arrangement located on the housing; the first flex indicator arrangement includes a first flex indicator that is configured to provide information to a user about a degree or amount of bending of the distal region of the flexible catheter prior, during and/or after the flex knob is rotated.

In another and/or alternative non-limiting object of the present disclosure is the provision of a system that delivers an expandable medical device to a treatment site wherein the first flex indicator arrangement includes a flex indicator window on an outer surface of the housing, and wherein the a first flex indicator is covered by the flex indicator window.

In another and/or alternative non-limiting object of the present disclosure is the provision of a system that delivers an expandable medical device to a treatment site wherein the first flex indicator includes a movable needle and a flex bend graphic; and wherein the needle moves relative to the flex bend graphic to indicate to a user a degree of bending of the distal region of the flexible catheter.

In another and/or alternative non-limiting object of the present disclosure is the provision of a system that delivers an expandable medical device to a treatment site wherein the first flex indicator is located rearwardly of the flex knob.

In another and/or alternative non-limiting object of the present disclosure is the provision of a system that delivers an expandable medical device to a treatment site further including a second flex indicator arrangement; the second flex indicator arrangement includes a second flex indicator; the second flex indicator is positioned on an opposite side of the housing from the first flex indicator.

In another and/or alternative non-limiting object of the present disclosure is the provision of a system that delivers an expandable medical device to a treatment site wherein the flex knob does not move along a longitudinal axis of the housing when the flex knob is moved on the housing.

In another and/or alternative non-limiting object of the present disclosure is the provision of a system that delivers an expandable medical device to a treatment site wherein the distal end of the flexible catheter includes a pull ring; the wire member is connected to the pull ring; the pull ring is spaced from a distal end of the flexible catheter.

In another and/or alternative non-limiting object of the present disclosure is the provision of a system that delivers an expandable medical device to a treatment site wherein a longitudinal axis of the wire member when connected to the pull ring is parallel to a longitudinal axis of a central cavity of the pull ring; the wire member is connected to an outer surface of the pull ring.

In another and/or alternative non-limiting object of the present disclosure is the provision of a system that delivers an expandable medical device to a treatment site wherein the distal region of the flexible catheter includes a flexible hypotube or spring; the flexible hypotube or spring is configured to a) bend when the distal region of the flexible catheter is caused to bend by the flex adjustment mechanism, and b) unbend when the flex adjustment mechanism allows the distal region of the flexible catheter to unbend from a bent position by the flex adjustment mechanism; the flexible hypotube or spring is positioned further from the distal end of the flexible catheter than the pull ring.

In another and/or alternative non-limiting object of the present disclosure is the provision of a system that delivers an expandable medical device to a treatment site wherein the pull ring is connected to a distal end of the flexible hypotube or spring.

In another and/or alternative non-limiting object of the present disclosure is the provision of a system that delivers an expandable medical device to a treatment site wherein the distal region of the flexible catheter includes the flexible hypotube; the flexible hypotube is a laser cut hypotube.

In another and/or alternative non-limiting object of the present disclosure is the provision of a system that delivers an expandable medical device to a treatment site wherein the wire member is positioned in a cavity of the flexible hypotube or spring and runs under a top surface of the flexible hypotube or spring along a longitudinal length of the flexible hypotube or spring.

In another and/or alternative non-limiting object of the present disclosure is the provision of a system that delivers an expandable medical device to a treatment site wherein a distal region of the flexible catheter includes an inner layer of material that includes a wire cavity; the wire cavity is configured to receive a portion of the wire member; the wire member is configured to move within the wire cavity.

In another and/or alternative non-limiting object of the present disclosure is the provision of a system that delivers an expandable medical device to a treatment site wherein the inner layer of material is a braided material layer that includes the wire cavity.

In another and/or alternative non-limiting object of the present disclosure is the provision of a system that delivers an expandable medical device to a treatment site wherein the inner layer of material is positioned rearwardly of flexible hypotube or spring that is located in a distal region of the flexible catheter.

In another and/or alternative non-limiting object of the present disclosure is the provision of a system that delivers an expandable medical device to a treatment site wherein the expandable medical device is a prosthetic heart valve.

In another and/or alternative non-limiting object of the present disclosure is the provision of a flexible catheter that includes a catheter cavity that is configured to receive at least a balloon catheter and wherein the balloon catheter is movable along a longitudinal length of the catheter cavity and is rotatable about a longitudinal axis of the catheter cavity; the catheter cavity runs along a central longitudinal axis of the flexible catheter and along 80-100% of a longitudinal length of the flexible catheter; the flexible catheter includes a) an outer layer, and wherein the outer layer forms an outer surface of the flexible catheter along 50-100% of the longitudinal length of the flexible catheter, and wherein the outer layer runs along a central longitudinal axis of the flexible catheter and along 80-100% of a longitudinal length of the flexible catheter; b) and inner layer, and wherein the inner layer runs along a central longitudinal axis of the flexible catheter and along 80-100% of a longitudinal length of the flexible catheter; c) a flexible inner layer that includes a wire cavity, and wherein the flexible inner layer is positioned between the inner layer and the outer layer, and wherein the flexible inner layer is spaced from a distal end of the flexible catheter, and wherein at least a portion of the flexible inner layer is located at a distal region of the flexible catheter; d) a flexible hypotube, and wherein the flexible hypotube is located between the inner layer and the outer layer, and wherein the flexible hypotube is positioned in the distal region of the flexible catheter, and wherein at least a portion of the flexible hypotube is positioned distal to the flexible inner layer; e) a pull ring that is positioned at a distal region of the flexible catheter, and wherein the pull ring is positioned at or distal to a distal end of the flexible inner layer, and wherein the pull ring is positioned at a distal location to the flexible inner layer, and wherein the flexible hypotube is positioned at or spaced proximal to the pull ring; and f) a wire member, and wherein the wire member extends along at least a portion of the longitudinal length of the flexible catheter, and wherein the wire member is at least partially positioned in the wire cavity of the flexible inner layer and is moveable within the wire cavity, and wherein a distal end of the wire member is connected to the pull ring; and wherein movement of the wire member toward a proximal end of the flexible catheter causes the flexible hypotube of bend and cause a distal region of the flexible catheter to bend.

In another and/or alternative non-limiting object of the present disclosure is the provision of a flexible catheter wherein the flexible hypotube is a laser cut hypotube; the hypotube includes a plurality of laser cut slots.

In another and/or alternative non-limiting object of the present disclosure is the provision of a flexible catheter wherein the flexible inner layer of material is a braided material layer.

In another and/or alternative non-limiting object of the present disclosure is the provision of a flexible catheter wherein the pull ring is connected to the distal end of the flexible hypotube.

In another and/or alternative non-limiting object of the present disclosure is the provision of a flexible catheter wherein the wire member passes through an outer surface of the outer layer at a location that is distal to the proximal end of the flexible catheter.

In another and/or alternative non-limiting object of the present disclosure is the provision of a flexible catheter further including a pull-wire jacket that is positioned in the wire cavity of the flexible inner layer, and wherein the wire member is located in a cavity of the pull-wire jacket, and wherein the wire member is movable within the cavity of the pull-wire jacket, and wherein the pull-wire jacket extends 60-100% of a longitudinal length of the wire cavity of the flexible inner layer.

In another and/or alternative non-limiting object of the present disclosure is the provision of a flexible catheter wherein the pull-wire jacket is formed of a flexible and low friction material.

In another and/or alternative non-limiting object of the present disclosure is the provision of a flexible catheter wherein the hypotube, the flexible inner layer, the wire member, and/or the pull ring are formed of stainless steel.

In another and/or alternative non-limiting object of the present disclosure is the provision of a flexible catheter wherein the outer layer includes polyether block amide (PEBA) thermoplastic elastomer.

These and other objects and advantages will become apparent to those skilled in the art upon reading and following the description taken together with the accompanying drawings.

### BRIEF DESCRIPTION OF THE DRAWINGS

The accompanying drawings, which are incorporated into and form a part of the specification, schematically illustrate one or more example implementations of the disclosed technology and, together with the general description given above and detailed description given below, serve to explain the principles of the disclosed subject matter, and wherein:

FIG. 1 depicts another exemplary, non-limiting delivery system comprising a non-limiting delivery handle, a flexible catheter and a portion of a balloon catheter extending outwardly from a distal end of the flexible catheter in accordance with the present disclosure.

FIG. 2 is a side view of the non-limiting delivery handle of the delivery system of FIG. 1.

FIG. 3A is a cross-sectional side view of the delivery handle of FIG. 2.

FIG. 3B is an enlarged cross-sectional side view of the non-limiting components of the flex adjustment mechanism located in the distal region of the delivery handle in accordance with the present disclosure.

FIG. 4 is a cross-sectional view of the distal region of the flexible catheter that does not include the balloon catheter in accordance with the present disclosure.

FIG. 5 are cross-sectional side views of several sections of the flexible catheter along different locations along the longitudinal length of the flexible catheter in accordance with the present disclosure.

FIGS. 6A and 6B illustrate a side view and a top view of a non-limiting pull ring and wire member connected to the pull ring in accordance with the present disclosure.

FIG. 7 illustrates an isometric view of a non-limiting hypotube that can be positioned in the distal region of the flexible catheter in accordance with the present disclosure.

FIGS. 8A and 8B illustrate a cross-sectional view and a top view a non-limiting pull ring and wire members that are connected to the distal end of the hypotube in accordance with the present disclosure.

FIG. 9 is a photo of a non-limiting arterial access path for the flexible catheter of the delivery system in accordance with the present disclosure through an aortic root.

### DETAILED DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

Example implementations of medical devices that can be used with the delivery system in accordance with the present disclosure include, but are not limited to, stent, prosthetic valve, prosthetic heart valve, TAVR valve, mitral valve replacement, tricuspid valve replacement, pulmonary valve replacement, LAA (left atrial appendage) closure device, etc. In one non-limiting implementation, the delivery system is configured to deliver a prosthetic valve to a treatment site (e.g., in a patient's heart valve, etc.). The delivery handle of the delivery system in accordance with the present disclosure includes: (i) a flex adjustment mechanism that is configured to facilitate the bending and/or flexing of the distal region of a flexible catheter and/or balloon catheter to facilitate in the movement of a portion of the flexible catheter and/or balloon catheter about around various bends or arches in a vascular system (e.g. aortic arch bend, etc.) during the delivery of medical device to a treatment site.

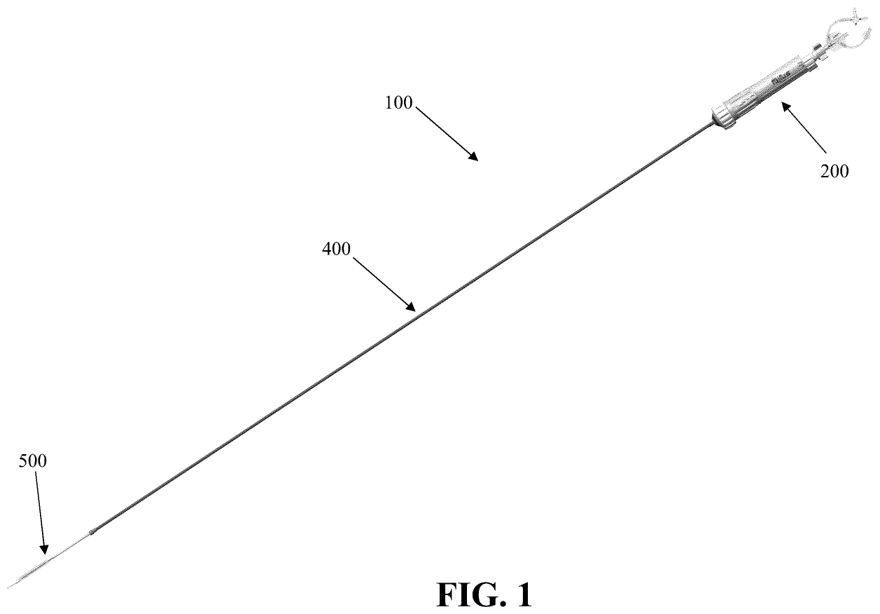

One or more implementations of the subject application will now be described with reference to the attached FIGS. 1-9.

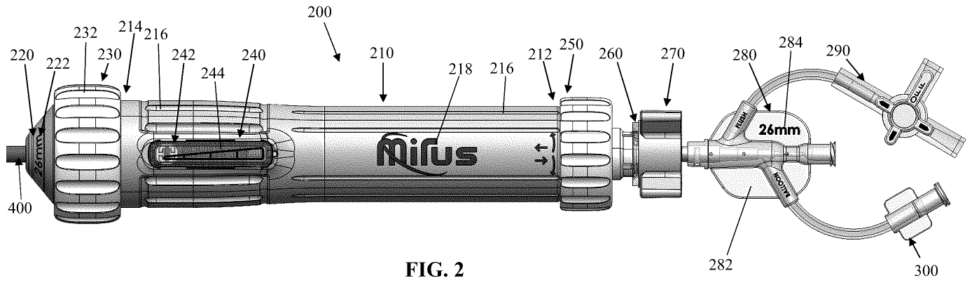

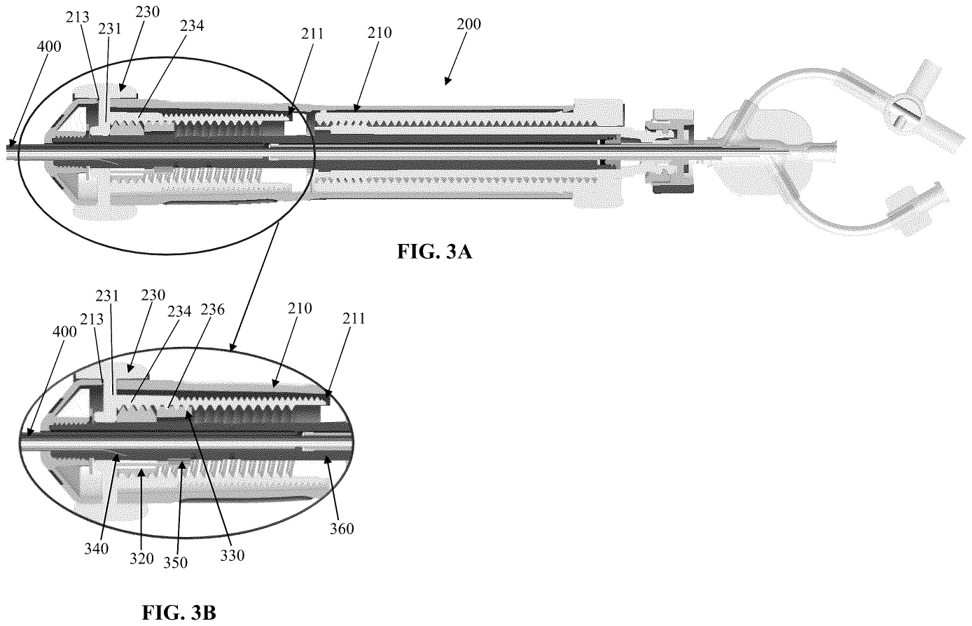

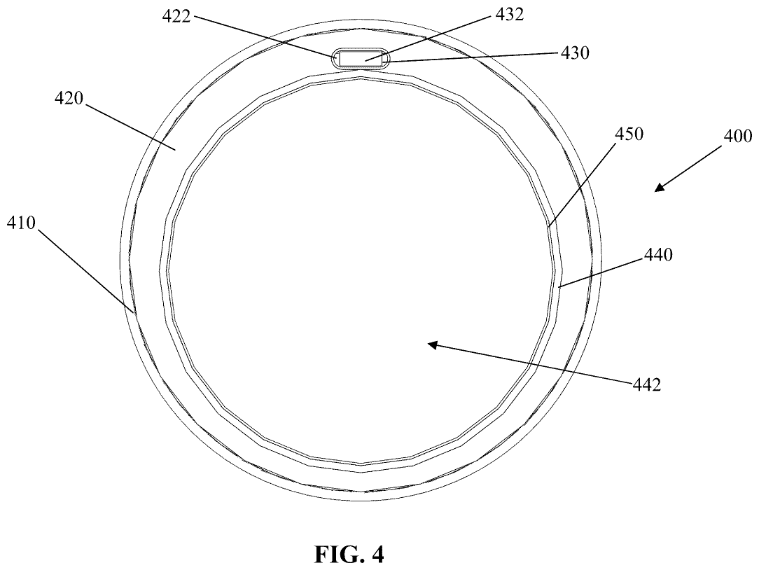

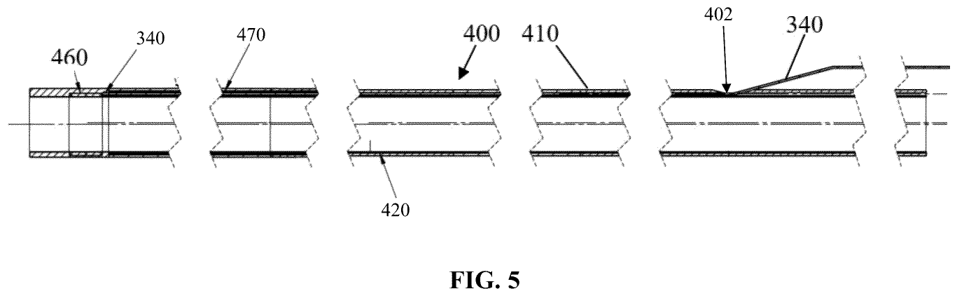

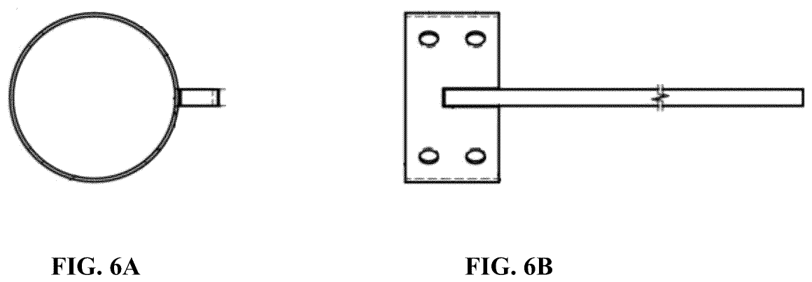

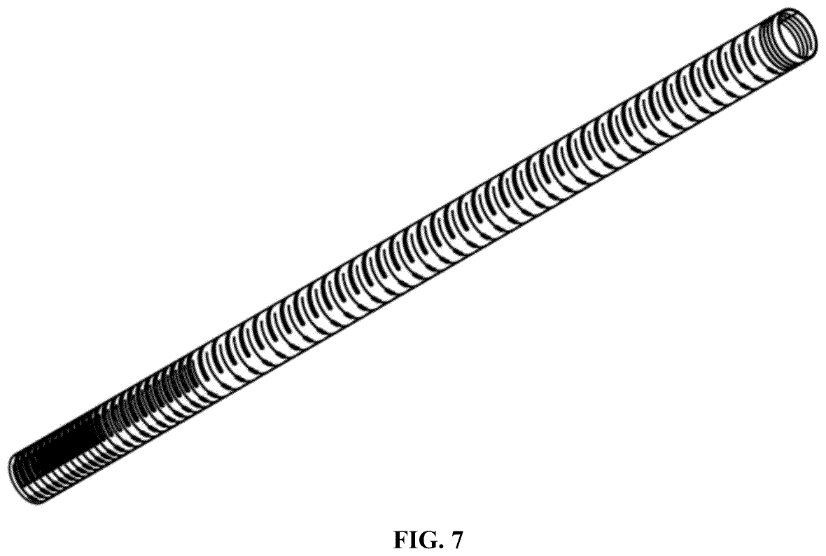

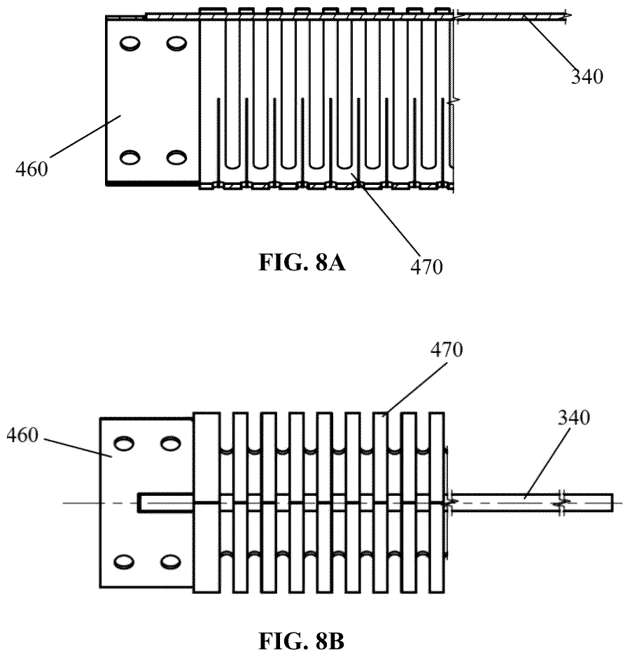

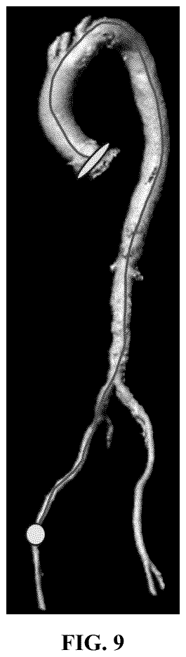

FIG. 1 depicts another exemplary, non-limiting delivery system comprising a non-limiting delivery handle, a flexible catheter and a portion of a balloon catheter extending outwardly from a distal end of the flexible catheter in accordance with the present disclosure. FIG. 2 is a side view of the non-limiting delivery handle of the delivery system of FIG. 1. FIG. 3A is a cross-sectional side view of the delivery handle of FIG. 2. FIG. 3B is an enlarged cross-sectional side view of the non-limiting components of the flex adjustment mechanism located in the distal region of the delivery handle in accordance with the present disclosure. FIG. 4 is a cross-sectional view of the distal region of the flexible catheter that does not include the balloon catheter in accordance with the present disclosure. FIG. 5 are cross-sectional side views of several sections of the flexible catheter along different locations along the longitudinal length of the flexible catheter in accordance with the present disclosure. FIGS. 6A and 6B illustrate a side view and a top view of a non-limiting pull ring and wire member connected to the pull ring in accordance with the present disclosure. FIG. 7 illustrates an isometric view of a non-limiting hypotube that can be positioned in the distal region of the flexible catheter in accordance with the present disclosure. FIGS. 8A and 8B illustrate a cross-sectional view and a top view a non-limiting pull ring and wire members that are connected to the distal end of the hypotube in accordance with the present disclosure. FIG. 9 illustrates a non-limiting arterial access path for the flexible catheter of the delivery system in accordance with the present disclosure through an aortic root.

Referring now to FIG. 1-8, the delivery system 100 includes a delivery handle 200, a flexible catheter 400 and a balloon catheter 500 or guide shaft (if not inflatable balloon is used) that is movable relative to the flexible catheter 400. An expandable prosthetic valve, not shown can be inserted about a portion or all of the distal end or distal region of the balloon catheter 500. The proximal end or region of the flexible catheter 400 is connected to the distal end or region of the delivery handle 200. A portion or all of the flexible catheter is flexible so that the distal region of the flexible catheter 400 can be inserted through a vascular system and to a treatment site of a patient. Various cross-sections of a portion of the flexible catheter 400 are illustrated in FIGS. 4, 5, 6A, 6B, 7, 8A and 8B and will be described in more detail below. The balloon catheter 500 includes an inflatable balloon positioned near the distal end of the ballon catheter (e.g., inflatable balloon is spaced from the end of the balloon catheter a distance of 0-5% [and all values and ranges therebetween] of the longitudinal length of the balloon catheter).

Referring now to FIGS. 2, 3A and 3B, a non-limiting delivery handle 200 in accordance with the present disclosure is illustrated. The delivery handle 200 is configured for use with a medical device such as, but not limited to, a prosthetic heart valve. The delivery handle 200 includes an elongated housing 210 that has a proximal region 212 and a distal region 214. The housing 200 is a generally circular cross-sectional shape along a longitudinal axis of the housing; however, other shapes can be used. The cross-sectional area of the housing 200 can optionally vary along the longitudinal length of the housing. For example, in the present non-limiting embodiment, the housing has a smaller cross-sectional area in the mid-region of the housing, as compared to the regions located at and/or near the proximal and distal ends. In the present non-limiting embodiment, the housing 200 has a length that is sufficient for a user to easily grasp the housing with the user's hand (e.g., 4-12 inches and all values and ranges therebetween). The housing 200 further includes a middle handle portion that has an outer surface. The outer surface can optionally include one or more optional gripping features 216 (e.g., ribs, etc.) to facilitate in the holding and handling of the delivery handle 200. The type, configuration and number of optional gripping features 216 are non-limiting.

The outer surface of the housing 210 can also optionally include one or more orientation markings 218 that can optionally be used by a user to indicate the proper orientation of the delivery handle 200 during use (e.g., proper commissural alignment of medical device at treatment site, proper commissural alignment of prosthetic heart valve at treatment site in heart, etc.). As illustrated in FIG. 2, there is a marking 218 in the form of a “MiRus” logo that is positioned on the handle. This marking can be used as a visual indicator that when the marking is facing upwardly on the handle 200, the handle 200 is in the proper position during a medical device insertion procedure. As can be appreciated, multiple marking can be positioned on the handle 200. The type and number and shape of the one or more marking 218 on the handle are non-limiting.

The housing 210 includes a nose cap 220 that is located at the distal end of the distal region 214 of the housing 210. The nose cap 220 optionally has a size indicator 222 on the outer surface of the nose cap 220 that can optionally provide size information about the flexible catheter and/or balloon catheter and/or provide size information about the medical device on the flexible catheter or balloon catheter.

The housing includes a flex knob 230 that is movably connected to the housing 210 and optionally moveably connected at the distal region 214 of the housing 210. The flex knob 230 is illustrated as being rotatable about a longitudinal axis of the housing; however, it can be appreciated that the flex knob can move in other ways (e.g., flex knob slides only a longitudinal axis of the housing, etc.). As can be appreciated, the flex knob can be located on other regions of the housing 210. As also can be appreciated, the flex knob 230 can be substituted by other components (e.g., flex switch, flex button, flex tab, electronic flex switch mechanism, etc.). As illustrated in FIG. 2, the flex knob optionally includes one or more grip features 232.

As illustrated in FIG. 2, the housing can also optionally include one or more other features such as a) one or more flex indicator windows 240 that are optionally located on the distal region 214 or other region of the housing 210, and wherein the orientation and configuration of the flex indicator window (e.g., a rectangular flex indicator window that has its longest length aligned with the longitudinal axis of the handle and wherein orienting the flex indicator window at some position relative to the top of the handle during use [e.g., orienting the flex indicator window to be on top or at 90° to the top of the handle during use, etc.]) can optionally be used by a user to indicate the proper orientation of the delivery handle 200 during use (e.g., proper commissural alignment of medical device at treatment site, proper commissural alignment of prosthetic heart valve at treatment site in heart, etc.), b) a flex indicator 242 that is optionally positioned behind the flex indicator window 240, c) an optional flex bend graphic 244 that is position on or inside the flex indicator window 240, and wherein the optional flex bend graphic provides information on the amount of bend or flex of the distal region of the flexible catheter and/or balloon catheter that is caused by use of the flex knob 230, d) a positioning wheel 250 that is configured to be movably connected to the distal region 214 of the housing 210, and wherein movement (e.g., rotational movement, sliding movement, etc.) of the positioning wheel causes fine longitudinal movement of the balloon catheter relative to the flexible catheter forwardly and rearwardly from the distal end of the housing 200, e) a hypotube that is located within the housing 200, and wherein the hypotube is movable within the housing along the longitudinal axis of the housing, and wherein a distal end of the hypotube is optionally connected to a proximal end of the balloon catheter, f) a positioning lock 270 that can be moved (e.g., rotated, slid, etc.) between a locked and unlocked position to lock or unlock the hypotube in the housing to control longitudinal movement of the hypotube relative to the housing, g) a luer tree 280 that is connected to proximal end of the hypotube, and wherein the luer tree is optionally rotatable about the longitudinal axis of the housing, and wherein rotation of the luer tree about the longitudinal axis of the housing optionally causes the hypotube and the balloon catheter to rotate so as to facilitate in commissural alignment of a medical device at the treatment site, and wherein the luer tree optionally includes one or more grasping tabs 282 or grasping features to enable a user to grasp the luer tree to cause the hypotube to be moved forwardly and rearwardly in the housing along a longitudinal axis of the housing, which optionally causes longitudinal movement of the balloon catheter at the treatment site, and wherein the optional one or more grasping tabs 282 or grasping features optionally enable a user to grasp the luer tree to rotate hypotube about the longitudinal axis of the housing, and wherein the luer tree includes one or more fluid ports, and wherein the luer tree optionally includes a size indicator 284 on the outer surface of the luer tree 290 that optionally provides size information about the flexible catheter and/or balloon catheter and/or provides size information about the medical device on the flexible catheter and/or balloon catheter, and/or h) a flush luer 290 and a balloon luer 300 that are connected to a port on the luer tree, wherein the flush luer 290 creates a port to flush the balloon catheter, and wherein the balloon luer 300 creates a port that can connect to an endoflator so as to enable expansion of the balloon on the flexible catheter and/or balloon catheter. As can be appreciated, the delivery handle can include other or additional features.

Referring again to FIG. 2, the housing 210 generally has a circular cross-sectional shape along a longitudinal axis of the housing. The cross-sectional area of the housing can optionally vary along the longitudinal length of the housing. For example, in the present non-limiting embodiment, the housing comprises a smaller cross-sectional area in the mid-region of the housing, as compared to the regions located at and/or near the distal end. In the present non-limiting embodiment, the housing has a length that is sufficient for a user to easily grasp the housing with the user's hands (e.g., 4-14 inches and all values and ranges therebetween).

As illustrated in FIGS. 3 and 3A, the flex adjustment mechanism is located near the distal end of the housing 210; however, the flex adjustment mechanism can be located on other regions of the housing. In one non-limiting embodiment as illustrated in FIGS. 2, 3 and 3A, the flex adjustment mechanism includes a flex knob 230 that is rotatably connected to the housing 210 such that the flex knob 230 rotates about a longitudinal axis (e.g., central longitudinal axis, etc.) of the housing 210. The flex knob 230 includes a threaded body portion 234 that is located in the cavity of housing 210. The flex knob 230 is configured such that is does not move longitudinally along the longitudinal axis of housing 210. As illustrated in FIGS. 3 and 3A, a vertical body flange 231 extends from the top portion of the flex knob into the interior cavity 211 of the housing 210 via a housing slot 213 and is connected to the threaded body portion 234. The engagement of the vertical body flange with the housing slot prevents longitudinal movement of the flex knob 230 along the longitudinal axis of the housing 210, and allows rotational movement of the flex knob about the longitudinal axis of the housing 210. The lower end of the vertical body flange 231 engages or is positioned closely adjacent to the body core 360 in the housing and moves about the outer surface of the body core 360 when the flex knob is rotated.

The body portion 234 of the flex knob 230 optionally includes a plurality of threads 236. Threads 236 are configured to engage flex insert 320 and/or flex actuator 330. As flex knob 230 is rotated by a user, the flex knob 230 causes direct or indirect the movement of the flex insert 320 and/or flex actuator 330 forwardly along the longitudinal axis of the housing 210 when the flex knob 230 is rotated in one direction, or cause rearward movement of the flex insert 320 and/or flex actuator 330 along the longitudinal axis of the housing 210 when the flex knob 230 is rotated in the opposite direction. As can be appreciated, the flex insert 320 and/or flex actuator 330 can be separate components or a single component. In one non-limiting arrangement, a) the flex insert 320 includes threading such that when the flex knob is rotated, the flex insert 320 remains in position and does not move along the longitudinal axis of the housing, and b) the flex actuator 330 includes threading such that when the flex knob is rotated, the flex actuator 330 is cause to move along the longitudinal axis of the housing, and wherein the wire member 340 is connected to the flex actuator.

The flex insert 320 and/or flex actuator 330 can optionally be connected to wire member 340 (e.g., pull-wire) by a clamp band 350 or other type of connection arrangement (e.g., weld, solder, adhesive, melted connection, clamp, etc.). The wire member 340 can optionally be inserted through an opening in the flexible catheter 400 on the proximal end or other region of the flexible catheter and is connected at the distal end of the wire member to a pull ring or other component that is located in the distal region of the flexible catheter 400. In one non-limiting arrangement, the distal region or distal end of the flexible catheter 400 includes a pull ring 460 which is connected to one end of the wire member 340.

FIGS. 6A and 6B illustrate a non-limiting pull ring 460 that can be located in the distal region of the flexible catheter. As discussed further below, the wire member 340 (e.g., flat wire, round cross-sectional wire, etc.) is illustrated as being welded to the outer surface of the pull ring 460.

When the flex knob 230 is rotated, the rotation of the flex knob 230 causes the flex insert 320 and flex actuator 330 to move either forwardly or rearwardly along the longitudinal axis of the housing 210, depending on the rotational direction of the flex knob. When the flex insert 320 and/or flex actuator 330 are caused to move rearwardly along the longitudinal axis of the housing 210, a portion of the wire member 340 is pulled into the housing. When the flex insert 320 and/or flex actuator 330 are caused to move forwardly along the longitudinal axis of the housing 210, a portion of the wire member 340 is pushed out of the housing. The pulling of the wire member 340 into the housing 210, causes controlled bending or flexing of the distal region of the flexible catheter and/or balloon catheter. The pushing of the wire member 340 out of the housing 210, causes unflexing or unbending or straightening of the distal region of the flexible catheter and/or balloon catheter. As such, the controlled rotation of the flex knob can be used to a) control when bending and/or straightening of the distal region of the flexible catheter and/or balloon catheter occurs, and/or b) control the amount of bending or flexing of the distal region of the flexible catheter and/or balloon catheter. In one non-limiting configuration, the flex knob 230 can be used to cause bending of the distal region of the flexible catheter and/or balloon catheter anywhere 0-360° (and all values and ranges therebetween) relative to the longitudinal axis of the proximal unbent or unflexed portion of the flexible catheter.

When the flex indicator 242 is a needle or other type of structure that is configured to move within indicator windows 240, the flex indicator 242 can be connected to the flex insert 320 and/or flex actuator 330 and be caused to move when the flex insert 320 and/or flex actuator 330 moved within the housing. In such an arrangement, when the flex insert 320 and/or flex actuator 330 move longitudinally along the longitudinal axis of housing 210, the flex indicator 242 is caused to move within indicator window 240 (e.g., move along the longitudinal axis of the housing, etc.) to provide visual information to a user about the amount of bending or flexing of the distal region of the flexible catheter and/or balloon catheter. As can be appreciated, when the flex indicator is in some other form (e.g., digital indicator, light indicator, sound indicator, dial, etc.), the movement of the flex insert 320 and flex actuator 330 can be used to cause the flex indicator to provide information on the amount of bending of the distal region of the flexible catheter and/or balloon catheter 400.

As illustrated in FIG. 2, a flex bend graphic 244 is position on or inside the flex indicator window 240. The flex bend graphic 244 provides information on the amount of bend or flex of the flexible catheter and/or balloon catheter caused by use of the flex knob 230. As illustrated in FIG. 2, the flex band marking 244 is in the form of a ramp wherein the ramp is thinnest toward the distal end of the housing and thickest toward the proximal end of the housing. The ramp has a constant slope; however, this is not required. The thinnest portion of the ramp represents little or no bending or flexing of the distal region of the flexible catheter and/or balloon catheter when the flex indicator 242 is positioned at the thinnest portion of the ramp. When the flex indicator 242 is positioned at the thickest portion of the ramp, such positioning of the flex indicator represents the maximum bending of the distal region of the flexible catheter and/or balloon catheter. As such, as the flex indicator moves along the flex band marking 244 in the form of a ramp marking, a user can view of the location of the flex indicator on the flex band marking and use such information to determine the relative amount of bending of the distal region of the flexible catheter and/or balloon catheter. As can be appreciated, other types of flex band marking 244 can be used (e.g., one or more bars, digital readout, digital graphics, electronic readout, one or more bend or flex symbols, one or more numbers, one or more percentage values, one or more bend angle values, etc.).

Referring now to FIGS. 4-5, there are illustrated various cross-sections of the distal region of the flexible catheter 400. FIG. 4 illustrates a non-limiting cross-section of the distal region of the flexible catheter 400. FIG. 5 illustrate a non-limiting cross-section of several side sections of the flexible catheter 400 along the longitudinal length of the flexible catheter 400. In both FIGS. 4 and 5, the balloon catheter 500 is not illustrated in the interior cavity of the flexible catheter 400.

The flexible catheter 400 includes a first or outer layer 410 and a cavity 442 that allows for other components pass along the longitudinal length of the flexible catheter (e.g., guide wire, hypotube 470, balloon catheter, other fluid tubes, etc.). The material that forms the outer layer is non-limiting. Generally, the material used to form the outer layer 410 is a flexible material. In one non-limiting embodiment, the outer layer is formed of a flexible polymer material such as, but not limited to, a polyether block amide (PEBA) thermoplastic elastomer (e.g., PEBAX™, etc.), polyester, polyethylene (PE), polypropylene (PP), polyvinyl chloride (PVC), thermoplastic polyurethane (TPU), etc. The outer layer 410 includes a cavity that allows for other components of the flexible catheter 400 to pass along the longitudinal length of the flexible catheter (e.g., optional braided layer 420, optional liner 400, optional additional liner 450, guide wire, hypotube 470, balloon catheter, other fluid tubes, etc.).

A second optional layer of the flexible catheter 400 is a braided layer 420. The braided layer, when used, is a flexible layer and can be formed of a metal material or non-metal material. The braided layer 420 generally extends 10-100% (and all values ad ranges therebetween) of the longitudinal length of the flexible catheter 400. The thickness of the braided layer 420 may or may not vary about the circumference of the flexible catheter 400. The braided layer 420 includes a cavity that allows for other components of the flexible catheter 400 to pass along the longitudinal length of the flexible catheter (e.g., optional liner 400, optional additional liner 450, guide wire, hypotube 470, balloon catheter, other fluid tubes, etc.). As best illustrated in FIG. 4, the thicker portion of braided layer 420 includes a pull-wire cavity 422 that is configured to form a channel through a portion or all of the longitudinal length of the flexible catheter 400 for wire member 340. The cross-sectional area or diameter of pull-wire cavity 422 is generally greater than the cross-sectional area or diameter of wire member 340 that is positioned in pull-wire cavity 422. The position of the pull-wire cavity 422, when used, is generally positioned closer to the bottom of the braided layer 420 (e.g., closer to the center of the flexible catheter) than to the top of the braided layer which is located closer to the outer layer. In one non-limiting configuration, the minimum thickness of the braided layer that is absent the pull-wire cavity (e.g., the thickness of the braided layer located diametrically opposite of the pull-wire cavity) is 70-150% (and all values and ranges therebetween) of the minimum thickness of a portion of the braided layer that is between the top edge of the pull-wire cavity and the top edge of the braided layer that includes the pull-wire cavity. As illustrated in FIG. 4, the positioning of the pull-wire cavity in the braided layer is such that the minimum thickness of the braided layer that is absent the pull-wire cavity (which minimum thickness location is generally the opposite side of the location of the pull-wire cavity) is about 95-105% of the minimum thickness between the top edge of the pull-wire cavity and the top edge of the braided layer that includes the pull-wire cavity.

The pull-wire cavity 422 can optionally include a cavity sleeve or pull-wire jacket 430 that has a sleeve cavity 432 that is positioned along 0.1-100% (and all values and ranges therebetween) the longitudinal length of the pull-wire cavity. The material used to form the cavity sleeve 430 is non-limiting. The shape of the cavity sleeve 430 is non-limiting. In one non-limiting embodiment, the cavity sleeve is formed of a flexible and low friction material (e.g., PTFE, etc.). When a cavity sleeve 430 is used, the cross-sectional area of the sleeve cavity 432 through the cavity sleeve 430 is generally greater than the cross-sectional area or diameter of wire member 340 that is positioned in the sleeve cavity 432. The cavity sleeve is generally configured to facilitate in the movement of the wire member 340 in the pull-wire cavity 422. When the cavity sleeve 430 is used, the wire member is positioned in the sleeve cavity 432.

As illustrated in FIG. 5, the wire member 340 can optionally exit the flexible catheter 400 prior to the proximal end of the flexible catheter via a wire opening 402. In such a non-limiting configuration, the wire member 340 can exit the flexible catheter 400 at the proximal end of the braided layer 420 in the flexible catheter, or at a location between the proximal and distal ends of the braided layer 420.

An optional third layer of the flexible catheter 400 is a liner 440. Liner 440 is generally formed of a flexible material. One non-limiting material for the liner 440 is a flexible and low friction material (e.g., PTFE, etc.). The liner 440 includes a cavity that allows for other components of the flexible catheter 400 to pass along the longitudinal length of the flexible catheter (e.g., optional additional liner 450, guide wire, hypotube 470, balloon catheter, other fluid tubes, etc.). The thickness of the second layer (braided layer) is illustrated as being a thicker layer than the outer layer 410 and liner 440. The outer layer 410 is generally thicker than liner 440; however, this is not required. Generally, the material used to form the second layer (braided layer) 420 is different from the material used to form the outer layer 410 and liner 440. Generally, the material used to form the outer layer 410 is different from the material used to form the liner 440.

An optional fourth layer of the flexible catheter 400 is a secondary liner or coating 450. The secondary liner or coating 450 can be a low friction liner or coating. Such secondary liner or coating 450 is illustrated as being the thinnest layer of the flexible catheter 400; however, this is not required. The secondary liner or coating 450 includes a cavity that allows for other components of the flexible catheter 400 to pass along the longitudinal length of the flexible catheter (e.g., guide wire, hypotube 470, balloon catheter, other fluid tubes, etc.).

Referring now to FIG. 5, several side cross-sections of the flexible catheter 400 are illustrated. The distal region of the flexible catheter 400 can include a pull ring 460 at or near the distal end of the flexible catheter 400. The shape and size of the pull ring is non-limiting. The pull ring 460 is generally fixed in location in the flexible catheter 400. The distal end of the wire member 340 is connected to the pull ring 460. As illustrated in FIG. 5, the pull ring 460 is spaced rearwardly from the distal end of the flexible catheter 400.

Positioned rearwardly of the pull ring 460 is a flexible hypotube 470. The shape, size and material of the hypotube 470 is non-limiting. FIG. 7 illustrates a non-limiting configuration of hypotube 470. FIGS. 8A and 8B illustrates a non-limiting connection (e.g., weld connection, solder connection, adhesive connection, etc.) of the pull ring 460 to the distal end of hypotube 470.

The hypotube 470 is typically only located in the distal region of the flexible catheter 400. The longitudinal length of the hypotube 470 is generally 1-25% (and all values and ranges therebetween) of the longitudinal length of the flexible catheter 400. The hypotube 470 can be formed of a shape memory material; however, this is not required. The hypotube 470, when used, facilitates in the bending and unbending of the distal region of the flexible catheter 400. When a braided layer 420 is used, the braided layer 420 can a) be located readwardly of (e.g., positioned at or spaced from) the proximal end of the hypotube 470, b) overlie a portion of the proximal region of the hypotube or c) located within a portion of the internal cavity of the hypotube. Generally, if the braided layer overlies a portion of the hypotube, the braided layer 420 does not completely overlie the complete longitudinal length of the hypotube. Also, if the braided layer is located in the internal cavity of the hypotube, the braided layer 420 generally does not extend along the complete longitudinal length of the internal cavity of the hypotube.

As illustrated in FIGS. 8A and 8B, the longitudinal length of the pull ring 460 is less than the longitudinal length of the hypotube.

Referring again to FIG. 7, the hypotube 470 includes a plurality of cut sections on the outer surface of the hypotube 470, wherein such cut sections facilitate in the bending of the hypotube 470 when the distal region of the flexible catheter is caused to be bent. The cut sections in the hypotube 470 can also be configured to facilitate in causing the hypotube to reform to its original unbent configuration when the distal region of the flexible catheter is allowed to move back to its fully or partially unbent configuration. The cut sections in the hypotube can also be formed to allow the hypotube to be bent at least 50° (e.g., at least 50° to at least 180° and all values and ranges therebetween) without damaging the hypotube 470 or causing the hypotube 470 to have a permanently bent form after being initially bent. The cut configuration in the hypotube is non-limiting.

Referring again to FIGS. 6A, 6B, 8A and 8B, the pull ring 460 is illustrated as being connected to the distal end of the hypotube 470. The type of connection is non-limiting (e.g., adhesive connection, weld, solder, melted connection, clamp or other mechanical connection, etc.). The wire member 340 is connected to the outer surface of the pull ring 460 such longitudinal axis of the wire member is generally parallel to the longitudinal axis of the cavity through the pull ring 460 as illustrated in FIG. 6B. As illustrated in FIGS. 8A and 8B, the wire member extends rearwardly from the pull ring and runs under the top surface of the hypotube 470. When a braided layer 420 is used and is located rearwardly of the hypotube, the wire member 340 then extends into the pull-wire cavity 422 of the braided layer 420 as discussed above. The distal region of the pull ring 460 can optionally include a tapered tip.

Referring again to FIG. 5, the flexible catheter 400, the longitudinal length of the proximal region of the flexible catheter 400 is generally longer than the distal region of the flexible catheter 400. In one non-limiting configuration, the proximal region of the flexible catheter 400 is generally 55-90% (and all values and ranges therebetween) of the longitudinal length of the flexible catheter 400. In one non-limiting configuration, the proximal region of the flexible catheter 400 is 75-85% of the longitudinal length of the flexible catheter. The longitudinal length of the hypotube is generally about 20-99.5% (and all values and ranges therebetween) of the longitudinal length of the distal region of the flexible catheter 400.

In one non-limiting configuration, the cross-sectional area of the distal region of the flexible catheter 400 that includes the hypotube is greater than the average cross-sectional area of the proximal region of the flexible catheter 400. The longitudinal length of the pull ring 460 is generally 0.5-5% (and all values and ranges therebetween) of the longitudinal length of the distal region of the flexible catheter 400. Generally, the pull ring is a non-flexible component of the flexible catheter, and is generally fixed in location in the flexible catheter. In one non-limiting configuration, the wire member 320, pull ring 460, hypotube 470 and/or the braided layer 420 can optionally be formed of the same materials (e.g., stainless steel, rhenium and/or hafnium alloy, etc.).

In operation, the flexible catheter 400 is inserted into a vascular passageway (e.g., blood vessel, etc.) until the distal region of the flexible catheter 400 reaches the treatment site. As illustrated in FIG. 9, the pathway to the treatment site is not always straight. The line illustrated in FIG. 9 illustrates a non-limiting pathway from the entry point in the vascular passageway to the treatment site. In regions of the vascular passageway where the pathway curves more than 20°, the proximal end of the flexible catheter 400 may damage the inner wall of the vascular passageway as the flexible catheter is moved through such curved pathway. The flex adjustment mechanism on the delivery system of the present disclosure can be used to inhibit or prevent such damage to the inner wall of the vascular passageway as the flexible catheter is moved through such curved pathway. When the distal end of the flexible catheter reaches the curved passageway, a user can move or rotate the flex knob 230 on the housing of the delivery handle so as to cause the wire member 340 to move within the flexible catheter 400. As discussed above, such movement of the wire member causes the distal region of the flexible catheter to bend and unbend. The user can control the amount of movement or rotation of the flex knob to obtain the desired bending of the flexible catheter. A user can rotate the delivery handle 210, which in turn causes the rotation of the flexible catheter 400. Such rotation of the flexible catheter 400 facilitates in orienting the flexible catheter in the vascular passage to obtain the desired bending direction of the distal region of the flexible catheter when the flex knob is moved or rotated. After the distal region of the flexible catheter has passed through a curved region in the vascular passageway, the user can move or rotate the flex knob to cause the distal region of the flexible catheter to unbend. As can be appreciated, the movement or rotation of the flex knob and/or the rotation of the flexible catheter by the delivery handle can be used to proper position the medical device at the treatment site (e.g., center the expandable medical device at the treatment site, ensure proper commissural alignment of the medical device at the treatment site, etc.).

It should be understood, however, that all of these and similar terms are to be associated with the appropriate physical quantities and are merely convenient labels applied to these quantities.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. These and other modifications of the preferred embodiments as well as other embodiments of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

The description of embodiments and examples has been presented for purposes of illustration and description. It is not intended to be exhaustive or limiting to the forms described. Numerous modifications are possible in light of the teachings herein. Some of those modifications have been discussed, and others will be understood by those skilled in the art. The embodiments were chosen and described in order to illustrate principles of various embodiments as are suited to particular uses contemplated. The scope is, of course, not limited to the examples set forth herein, but can be employed in any number of applications and equivalent devices by those of ordinary skill in the art.

To aid the Patent Office and any readers of this application and any resulting patent in interpreting the claims appended hereto, applicants do not intend any of the appended claims or claim elements to invoke 35 U.S.C. 112 (f) unless the words “means for” or “step for” are explicitly used in the particular claim.

What is claimed:

1. A delivery handle arrangement that facilitates a delivery of a medical device to a treatment site; said delivery handle arrangement comprising:
a housing having an inner core that at least partially extends linearly between a proximal end and a distal end of said housing;
a flexible catheter that is connected to said housing; and
a flex adjustment mechanism that is configured to controllably bend a distal region said flexible catheter; said flex adjustment mechanism includes:
a flex knob that is movably connected to said housing; said flex knob includes a body portion that is at least partially positioned in said inner core of said housing;
a flex insert and/or flex actuator that is at least partially positioned in said inner core of said housing; said flex insert and/or flex actuator is configured to move within said housing during movement of said flex knob; and
a wire member; said wire member is coupled at a proximal end to said flex insert and/or flex actuator and a distal region of said flexible catheter; and
wherein said distal region of said flexible catheter is caused to controllably bend during movement of said flex knob.

2. The delivery handle arrangement as defined in claim 1, wherein said flex knob is configured to rotate on said housing; said flex knob is configured to cause a portion of said wire member to be drawn into said housing when rotated in a first direction, and wherein said distal region of said flexible catheter is caused to bend when said flex knob is rotated in said first direction; said flex knob is configured to cause said wire member to be moved outwardly from said housing when rotated in a second direction, and wherein said distal region of said flexible catheter is caused to unbend when said flex knob is rotated in said second direction.

3. The delivery handle arrangement as defined in claim 1, further including a first flex indicator arrangement located on said housing; said first flex indicator arrangement includes a first flex indicator that is configured to provide information to a user about a degree or amount of bending of said distal region of said flexible catheter prior, during and/or after said flex knob is rotated.

4. The delivery handle arrangement as defined in claim 3, wherein said first flex indicator arrangement includes a flex indicator window on an outer surface of said housing, and wherein said a first flex indicator is covered by said flex indicator window.

5. The delivery handle arrangement as defined in claim 3, wherein said first flex indicator includes a movable needle and a flex bend graphic; and wherein said needle moves relative to said flex bend graphic to indicate to a user a degree of bending of said distal region of said flexible catheter.

6. The delivery handle arrangement as defined in claim 3, wherein said first flex indicator is located rearwardly of said flex knob.

7. The delivery arrangement as defined in claim 3, further including a second flex indicator arrangement; said second flex indicator arrangement includes a second flex indicator; said second flex indicator is positioned on an opposite side of said housing from said first flex indicator.

8. The delivery handle arrangement as defined in claim 1, wherein said flex knob does not move along a longitudinal axis of said housing when said flex knob is moved on said housing.

9. The delivery handle arrangement as defined in claim 1, wherein said distal end of said flexible catheter includes a pull ring; said wire member is connected to said pull ring; said pull ring is spaced from a distal end of said flexible catheter.

10. The delivery handle arrangement as defined in claim 9, wherein a longitudinal axis of said wire member when connected to said pull ring is parallel to a longitudinal axis of a central cavity of said pull ring; said wire member is connected to an outer surface of said pull ring.

11. The delivery handle arrangement as defined in claim 9, wherein said distal region of said flexible catheter includes a flexible hypotube or spring; said flexible hypotube or spring is configured to a) bend when said distal region of said flexible catheter is caused to bend by said flex adjustment mechanism, and b) unbend when said flex adjustment mechanism allows said distal region of said flexible catheter to unbend from a bent position by said flex adjustment mechanism; said flexible hypotube or spring is positioned further from said distal end of said flexible catheter than said pull ring.

12. The delivery handle arrangement as defined in claim 11, wherein said pull ring is connected to a distal end of said flexible hypotube or spring.

13. The delivery handle arrangement as defined in claim 11, wherein said distal region of said flexible catheter includes said flexible hypotube; said flexible hypotube is a laser cut hypotube.

14. The delivery handle arrangement as defined in claim 11, wherein said wire member is positioned in a cavity of said flexible hypotube or spring and runs under a top surface of said flexible hypotube or spring along a longitudinal length of said flexible hypotube or spring.

15. The delivery handle arrangement as defined in claim 1, wherein a distal region of said flexible catheter includes an inner layer of material that includes a wire cavity; said wire cavity is configured to receive a portion of said wire member; said wire member is configured to move within said wire cavity.

16. The delivery handle arrangement as defined in claim 15, wherein said inner layer of material is a braided material layer that includes said wire cavity.

17. The delivery handle arrangement as defined in claim 15, wherein said inner layer of material is positioned rearwardly of flexible hypotube or spring that is located in a distal region of the flexible catheter.

18. The delivery handle arrangement as defined in claim 1, wherein said expandable medical device is a prosthetic heart valve.

19. A system that delivers an expandable medical device to a treatment site, the system comprising:
a flexible catheter;
and
a delivery handle arrangement; said delivery handle arrangement comprising:
a housing having an inner core that at least partially extends linearly between a proximal end and a distal end of said housing;
a flex adjustment mechanism that is configured to controllably bend a distal region said flexible catheter; said flex adjustment mechanism includes:
a flex knob that is movably connected to said housing; said flex knob includes a body portion that is at least partially positioned in said inner core of said housing;
a flex insert and/or flex actuator that is at least partially positioned in said inner core of said housing; said flex insert and/or flex actuator is configured to move within said housing during movement of said flex knob; and
a wire member; said wire member is coupled at a proximal end to said flex insert and/or flex actuator and a distal region of said flexible catheter; and
wherein said flexible catheter that is connected to said housing; and
wherein said distal region of said flexible catheter is caused to controllably bend during movement of said flex knob; and
wherein movement of said flex knob in a first direction causes said flex insert and/or flex actuator to translate linearly along a longitudinal axis of said housing to cause movement of said wire member to cause said flexible catheter to controllably bend said flexible catheter.

20. The system as defined in claim 19, further including a first flex indicator arrangement located on said housing; said first flex indicator arrangement includes a first flex indicator that is configured to provide information to a user about a degree or amount of bending of said distal region of said flexible catheter prior, during and/or after said flex knob is rotated.

21. The system as defined in claim 20, wherein said first flex indicator arrangement includes a flex indicator window on an outer surface of said housing, and wherein said a first flex indicator is covered by said flex indicator window.

22. The system as defined in claim 20, wherein said first flex indicator includes a movable needle and a flex bend graphic; and wherein said needle moves relative to said flex bend graphic to indicate to a user a degree of bending of said distal region of said flexible catheter.

23. The system as defined in claim 20, further including a second flex indicator arrangement; said second flex indicator arrangement includes a second flex indicator; said second flex indicator is positioned on an opposite side of said housing from said first flex indicator.

24. The system as defined in claim 19, wherein said distal end of said flexible catheter includes a pull ring; said wire member is connected to said pull ring; said pull ring is spaced from a distal end of said flexible catheter; a longitudinal axis of said wire member when connected to said pull ring is parallel to a longitudinal axis of a central cavity of said pull ring; said wire member is connected to an outer surface of said pull ring.

25. The system as defined in claim 24, wherein said distal region of said flexible catheter includes a flexible hypotube or spring; said flexible hypotube or spring is configured to a) bend when said distal region of said flexible catheter is caused to bend by said flex adjustment mechanism, and b) unbend when said flex adjustment mechanism allows said distal region of said flexible catheter to unbend from a bent position by said flex adjustment mechanism; said flexible hypotube or spring is positioned further from said distal end of said flexible catheter than said pull ring; said pull ring is connected to a distal end of said flexible hypotube or spring.

26. A flexible catheter that includes a catheter cavity that is configured to receive at least a balloon catheter and wherein the balloon catheter is movable along a longitudinal length of said catheter cavity and is rotatable about a longitudinal axis of said catheter cavity; said catheter cavity runs along a central longitudinal axis of said flexible catheter and along 80-100% of a longitudinal length of said flexible catheter; said flexible catheter includes a) an outer layer, and wherein said outer layer forms an outer surface of said flexible catheter along 50-100% of said longitudinal length of said flexible catheter, and wherein said outer layer runs along a central longitudinal axis of said flexible catheter and along 80-100% of a longitudinal length of said flexible catheter; b) and inner layer, and wherein said inner layer runs along a central longitudinal axis of said flexible catheter and along 80-100% of a longitudinal length of said flexible catheter; c) a flexible inner layer that includes a wire cavity, and wherein said flexible inner layer is positioned between said inner layer and said outer layer, and wherein said flexible inner layer is spaced from a distal end of said flexible catheter, and wherein at least a portion of said flexible inner layer is located at a distal region of said flexible catheter; d) a flexible hypotube, and wherein said flexible hypotube is located between said inner layer and said outer layer, and wherein said flexible hypotube is positioned in said distal region of said flexible catheter, and wherein at least a portion of said flexible hypotube is positioned distal to said flexible inner layer; e) a pull ring that is positioned at a distal region of said flexible catheter, and wherein said pull ring is positioned at or distal to a distal end of said flexible inner layer, and wherein said pull ring is positioned at a distal location to said flexible inner layer, and wherein said flexible hypotube is positioned at or spaced proximal to said pull ring; and f) a wire member, and wherein said wire member extends along at least a portion of said longitudinal length of said flexible catheter, and wherein said wire member is at least partially positioned in said wire cavity of said flexible inner layer and is moveable within said wire cavity, and wherein a distal end of said wire member is connected to said pull ring; and wherein movement of said wire member toward a proximal end of said flexible catheter causes said flexible hypotube of bend and cause a distal region of said flexible catheter to bend.

27. The flexible catheter as defined in claim 26, wherein said flexible catheter includes one or more of a) said flexible hypotube is a laser cut hypotube; b) said hypotube includes a plurality of laser cut slots; said flexible inner layer of material is a braided material layer, c) said pull ring is connected to said distal end of said flexible hypotube, d) said wire member passes through an outer surface of said outer layer at a location that is distal to said proximal end of said flexible catheter, e) a pull-wire jacket that is positioned in said wire cavity of said flexible inner layer, and wherein said wire member is located in a cavity of said pull-wire jacket, and wherein said wire member is movable within said cavity of said pull-wire jacket, and wherein said pull-wire jacket extends 60-100% of a longitudinal length of said wire cavity of said flexible inner layer; f) said flexible inner layer, said wire member, and/or said pull ring are formed of stainless steel, and/or g) said outer layer includes polyether block amide (PEBA) thermoplastic elastomer.
